(19) United States
(12) Patent Application Publication
(10) Pub. No.: US 2020/0384158 A1
(43) Pub. Date: Dec. 10, 2020

(54) COMPOSITIONS AND METHODS FOR URINARY BLADDER REGENERATION

(71) Applicant: Northwestern University, Evanston, IL (US)

(72) Inventors: Arun Sharma, Elmwood Park, IL (US); Partha Hota, Rochester Hills, MI (US); Natalie Fuller, Chicago, IL (US); Derek Matoka, Chicago, IL (US); Earl Cheng, Elmhurst, IL (US)

(21) Appl. No.: 16/820,281

(22) Filed: Mar. 16, 2020

Related U.S. Application Data
(63) Continuation of application No. 15/652,983, filed on Jul. 18, 2017, now abandoned.
Application No. 15/652,983 is a continuation of application No. 14/548,079, filed on Nov. 19, 2014, now abandoned.
Application No. 14/548,079 is a continuation of application No. 12/816,780, filed on Jun. 16, 2010, now abandoned.
(60) Provisional application No. 61/187,489, filed on Jun. 16, 2009.
(60) Provisional application No. 61/241,201, filed on Sep. 10, 2009.

(51) Int. Cl. A61L 27/38; A61K 35/28; C12N 11/08; A61K 35/44; A61L 27/18; C12N 5/071

(52) U.S. Cl. CPC A61L 27/3886; A61K 35/28; C12N 11/08; C12N 2502/1352; A61L 27/18; C12N 5/0692; A61L 2430/22; A61K 35/44

(57) ABSTRACT

The present invention provides compositions and methods for the regeneration of tissue. In particular, the present invention provides mesenchymal stem cells (MSCs) and endothelial progenitor cells (EPCs) for bladder regeneration.

### CROSS-REFERENCE TO RELATED APPLICATION

The present application is a continuation of U.S. patent application Ser. No. 15/652,983, filed Jul. 18, 2017, which is a continuation of U.S. patent application Ser. No. 14/548,079, filed Nov. 19, 2014, now abandoned, which is a continuation of U.S. patent application Ser. No. 12/816,780, filed Jun. 16, 2010, now abandoned, which claims priority to U.S. Provisional Application 61/187,489, filed Jun. 16, 2009, and U.S. Provisional Application 61/241,201, filed Sep. 10, 2009, each of which are herein incorporated by reference in their entireties.

### FIELD OF THE INVENTION

The present invention provides compositions and methods for the regeneration of tissue. In particular, the present invention provides mesenchymal stem cells (MSCs) and endothelial progenitor cells (EPCs) for bladder regeneration.

### BACKGROUND

The urinary bladder is a muscular, hollow distensible organ which acts as a reservoir for biological waste which is ultimately expelled from the body through a highly orchestrated chain of physiological events. Patients suffering with congenital abnormalities including spina bifida exhibit a neuropathic bladder which causes a severe disruption in the events that allow for complete micturation. The neuropathic bladder is associated with poor compliance, urinary incontinence, infections, and the potential for renal failure if left untreated. Since the neuropathic bladder can be the result of developmental defects, conventional surgical management in which detubularized bowel is utilized as a patch (enterocystoplasty) is typically utilized. This procedure facilitates an increase in bladder volume by increasing the size of the bladder which results in decreased leak point pressures and an increase in compliance. This relieves pressure buildup upon the bladder and renal system during pre- and post-micturation processes. Although enterocystoplasty provides functional improvement, it is associated with significant complications including infection, stone formation, acute intestinal obstruction due to adhesion, increased mucus production, and the potential for malignant transformation (Bankhead et al. J Child Neurol 2000;15(3):141-149.; Cetinel. Adv Exp Med Biol 2003; 539(Pt A):509-533.; herein incorporated by reference in their entireties). Alternative methods to enterocystoplasty have been explored through tissue engineering by seeding cultured bladder cells on bioscaffolds as an augmentation patch. With these techniques, some partial success has been noted but regeneration of fully functional normal bladder tissue has yet to be achieved. Several obstacles currently limit the advancement in this research field including the choice of appropriate cell types for seeding, inadequate neovascularization of the seeded graft, and primitive bioscaffold design. Thus, alternative cell sources and advancements in bioscaffold design are needed. Epitope defined bone marrow derived MSCs represent a highly characterized population of cells that may be used to reconstitute the smooth muscle component of the dysfunctional bladder. Tissue regenerative studies implemented to recreate the native urinary bladder milieu have mainly been examined through the use of two matrix based strategies: “cell seeded” and “unseeded” technologies. The unseeded strategy relies upon the in-growth of native cells to regenerate the bladder onto an implanted biodegradable matrix. Unseeded technology has been hindered by scaffold contraction, variable degrees of regeneration, and a limited surface area of regenerated tissue which may be due to lack of vascularity and/or variability in the scaffold material itself (Metwalli et al. Curr Urol Rep. 4:156, 2003, herein incorporated by reference in its entirety). Alternatively, cell seeded strategies have been demonstrated by several groups with varying results. The regenerative process has been induced utilizing small intestinal submucosa (SIS) seeded with human bladder smooth muscle cells (SMCs) and urothelial cells (UCs) when implanted subcutaneously into the flanks of nude mice (Zhang et al. Tissue Eng. 10:181, 2004, herein incorporated by reference in its entirety). However, due to the inherent heterogeneous nature of biological material such as SIS, experimental applications are inconsistent. The bladder regenerative potential of synthetic polymers seeded with autologous sources of UCs and SMCs within the context of a canine bladder augmentation model has been demonstrated (Jayo et al. Regen Med. 3:671, 2008, herein incorporated by reference in its entirety). Data from these studies suggest that ex vivo expanded UCs and bladder SMCs can be utilized for the de novo formation of functional bladder tissue within canine models. These studies have had limited physiological utility when translated to patients with neurogenic bladder (Atala et al. Lancet. 367:1241, 2006, herein incorporated by reference in its entirety). Concerns remain regarding the application of pathologic cells which include disease reformation and lack of contractility in newly constructed bladders.

[symbol]

### SUMMARY

The present invention provides compositions and methods for the regeneration of tissue. In particular, the present invention provides mesenchymal stem cells (MSCs) and endothelial progenitor cells (EPCs) for bladder regeneration.

Bone marrow stem and progenitor cells (BMSPCs) represent highly defined populations of cells that maybe useful for bladder regeneration. BMSPCs are non-exclusively comprised of mesenchymal stem cells (MSCs) and endothelial progenitor cells (EPCs) which can be isolated from patients in a simple manner and expanded to large numbers (Pittenger et al. Science. 284:143, 1999, Smadja et al. J Cell Mol Med. 11:1149, 2007, herein incorporated by reference in their entireties). Certain developmental disease states appear to have no deleterious effects on BMSPC functionality (Isaikina et al. Exp Oncol. 28:146, 2006, herein incorporated by reference in its entirety).

MSCs represent a multipotent population of cells that are derived from a number of sources including bone marrow (BM) which can be terminally differentiated into cells including osteoblasts, adipocytes, and chondrocytes (Kern et al. Stem Cells. 24:1294, 2006, Pittenger et al. Science. 284:143, 1999, herein incorporated by reference in their entireties). Regenerating bladder tissue typically lacks functional vascularization that is required to nourish developing tissue and is often enveloped in omentum following enterocystoplasty in an attempt to enhance vascularization. BM EPCs have been demonstrated to form functional vasculature in vivo (Nishimura. EXS. 94:147, 2005, herein incorporated by reference in its entirety), thus providing a unique opportunity to utilize EPCs in a bladder regenerative setting. EPCs can be isolated simultaneously along with MSCs using cell surface markers and stimulated with cytokines to embark upon a terminal differentiation program to become vasculature.

In some embodiments, the present invention provides compositions and methods employing messenchymal stem cells (MSCs). In some embodiments, the present invention provides compositions and methods employing endothelial progenitor cells (EPCs). In some embodiments, the present invention provides compositions and methods employing MSCs and EPCs. For example, the present invention provides compositions comprising a cell mixture, said mixture comprising isolated MSCs and/or EPCs. In some embodiments a cell mixture comprising MSCs and/or EPCs is isolated from the bone marrow. In some such embodiments, the EPCs and/or MSCs are adult stem cells. The present invention is not limited by the source organism of the stem cells. In some embodiments, the source organism is mammal (e.g. human). In some embodiments, the composition comprises an in vitro cell culture. In some embodiments, the stem cells are self-renewable (e.g., in vitro or in vivo). In some embodiments, the stem cells of the present invention are multipotent.

In some embodiments, the present invention provides a cell mixture comprising MSCs and/or EPCs. In some embodiments, a cell mixture comprises MSCs and/or EPCs, and a thin-film scaffold. In some embodiments, the present invention provides isolated MSCs and/or EPCs. In some embodiments, isolated MSCs and/or EPCs are adherent to a thin-film scaffold. In some embodiments, a thin-film scaffold comprises poly(1,8 octanediol-co-citrate) (POC).

In some embodiments, the present invention provides methods for using MSCs and/or EPCs. For example, the present invention provides a method for transplanting stem cells in vivo, comprising the steps of 1) providing: a host organism and isolated MSCs and/or EPCs; and transplanting the stem cells into a tissue of the host. In some preferred embodiments, the MSCs and/or EPCs are transplanted into an injured or diseased tissue of the host. In some embodiments, the stem cells that are transplanted into the host are derived from the host (i.e., auto-transplantation). In some embodiments, isolated MSCs and/or EPCs are adherent to a thin-film scaffold. In some embodiments, a thin-film scaffold comprises POC. In some embodiments, transplanting comprises transplanting a thin-film scaffold and the adherent bone marrow stem and progenitor cells.

In some embodiments, the present invention provides methods for enriching a population of uncultured MSCs and/or EPCs. In some embodiments, the present invention provides methods for enriching a population of uncultured MSCs and/or EPCs, comprising the steps of: a) providing uncultured cells from bone marrow, the tissue containing MSCs and/or EPCs; b) contacting the uncultured cells with a reagent that selectively binds to said MSCs and/or EPCs (positive marker) or selectively binds cells other than MSCs and/or EPCs (negative marker); and c) selecting cells that bind to the positive reagent or selecting cells that do not bind to the negative reagent, wherein selected cells are enriched in MSCs and/or EPCs as compared with the uncultured cells. Cells can be selected using either positive or negative markers alone, or in combination. In preferred embodiments, the selected cells are enriched at least 2-fold (e.g., at least 10-fold, 20-fold, 100-fold, etc.) in MSCs and/or EPCs as compared with said uncultured cells. In some preferred embodiments, the reagent is an antibody. In some embodiments, the selecting step uses flow-cytometry. In yet other embodiments, the method further comprises the step of d) transplanting the selected cells into a host.

In some embodiments, the present invention provides methods for screening biological agents that affect proliferation, differentiation, gene expression, or survival of MSCs and/or EPCs, comprising the steps of: 1) preparing a cell culture of MSCs and/or EPCs; 2) contacting the MSCs and/or EPCs with at least one test compound, and 3) determining if the test compound has an effect on proliferation, differentiation, gene expression, or survival of the MSCs and/or EPCs (e.g., determining if the test compound enhances the ability of the stem cell to provide a therapeutic effect). The present invention is not limited by the nature of the test compound. In some embodiments, the test compound is a small molecule drug or a growth factor. In some embodiments, the screening method is carried out in vitro (e.g., in culture). In other embodiments, the screening method is carried out in vivo (e.g., in a host containing native or transplanted MSCs and/or EPCs).

### DESCRIPTION OF FIGURES

The specification may be better understood when read in conjunction with the accompanying drawings which are included by way of example and not by way of limitation.

FIG. 1 shows Western Blot Analyses of Known Bladder Smooth Muscle Cell Contractile Proteins. Lane 1: MSC, Lane 2: SMC control. Smooth muscle α-actin shows strong and moderate expressions in SMC control and MSCs respectively. Calponin antibody recognizes calponin 1/2/3 and demonstrates that calponin 1 and 3 are expressed in MSCs while only calponin 1 is seen in SMC control. Vimentin, transgelin, and caldesmon are abundantly expressed as compared to SMC control. The antibody for smoothelin detects various isoforms of smoothelin that range from 57 to 94 kD. Two isoforms (smoothelin A and B) are expressed in SMCs and MSCs.

FIGS. 2A-G show MSC Immunofluorescence Staining: A) Caldesmon (Alexa Red) and Myosin heavy chain (FITC), B) Calponin (Alexa Red) and α-smooth muscle actin (FITC), C) Desmin (Alexa Red) and Vimentin (FITC), D) Transgelin (Alexa Red) Smoothelin (FITC). DAPI was utilized to distinguish cell nuclei. E-G are control antibody staining samples for the aforementioned antibodies. (40× magnification; scale bar=20 μm).

FIG. 3 shows a histogram of the results of a cell proliferation assay. Although cell numbers were higher for bladder SMCs in SMC media, both MSCs and SMCs demonstrated greater growth at earlier (days 1-7) versus later (days 8-15) time-points.

FIG. 4 shows a histogram of an intracellular calcium release assay. 10 mM carbachol treatment resulted in 8.6±2.5 RFU (SMCs) and 5.8±0.8 RFU (MSCs) increases. Although there was an increase in RFUs for both populations there was no significant difference between groups with regard to magnitude of increase.

FIG. 5 shows confocal microscopic imaging of MSC contraction. The filmstrip depicts frame by frame confocal microscopic imaging of carbachol stimulated MSCs. The peak calcium intensity (exhibited by increase in fluorescence) occurred within 20 s of carbachol addition accompanied by physical cellular contractions within a 60 s time frame.

FIG. 6 shows images of morphological changes of EPCs undergoing differentiation: Panel A) spheroid-like EPCs at day 4 of cytokine treatment, Panel B) flattened and spindle-shaped appearing cells after prolonged cytokine treatment at day 14, Panel C) HUVEC2 control cell population at 14 days of culture. (20× Magnification)

FIG. 7 shows a photographic image of EPC seeded chicken chorioallantoic membrane (CAM) model. Pro-angiogenic environment supports growth of sorted bone marrow derived GFP labeled EPCs resulting in a neo-vasculature depicted within the arrows.

FIG. 8 shows CAM EPC derived tissue. Tissue was taken directly from CAM model and placed under fluorescent microscope and visualized. Other vessels (flanked by arrows) in the background appear black suggesting the GFP tissue is not due to autofluorescence.

FIG. 9 shows MHC I antibody staining of CAM derived EPC tissue: Panel A) positive staining with human specific MHC I antibody (not crossreactive with chicken species), Panel B) negative antibody control. Data indicates that CAM derived tissue is of human origin.

FIG. 10 shows in vitro terminal differentiation of SB MSCs: (Panel A) negative control samples for adipogenic differentiation; (Panel B) SB BM derived CD29+, CD44+, CD105+, CD169+ flow cytometry sorted MSCs that have been forcibly differentiated into adipocytes that are evident by the presence of Oil-Red positive lipid laden cells; (Panel C) negative control samples for osteogenic differentiation; (Panel D) osteogenic differentiation of SB derived MSCs and shows the presence of alizarin stained calcium deposits.

FIG. 11 shows trichrome staining of 10 week rat bladders augmented with MSC/Urotsa POC Films: (Panel A) seeded POC sample shows a very frail and thin layer of tissue, mostly devoid of cells with a fibrotic capsule; (Panel B) higher magnification shows high collagen content suggesting fibrosis; (Panel C/D) bladder SMC sample shows muscle organization but with a low overall measurable levels of muscle to collagen ratio suggesting a lower level of regeneration; (Panel E) adult MSC sample shows thickened layer of muscle wall comprised of approximately a 1:1 ratio of muscle to collagen; (Panel F) higher magnification indicates many areas in which smooth muscle bundle formation occurred with the regenerated area; (Panel G/H) SB patient derived MSC seeded POC films demonstrated robust trilayer regeneration with smooth muscle bundle formation. All trichrome images were taken in the region of the MSC/Urotsa POC film implantation. These areas were more robust than that found in the native bladder tissue. This preliminary data suggests that MSCs from patients with SB or adult population can be used in a bladder regenerative setting.

FIG. 12 shows immunohistochemical staining of 10 week Rat Bladders Augmented with MSC/Urotsa POC Films: (A) Adult MSC/POC films; (B) SB MSC/POC films. I=calponin (Alexa Red)/γ-tubulin (FITC); II=caldesmon (Alexa Red)/γ-tubulin (FITC); III=Human Specific Elastin (Alexa Red; IV=Human Specific γ-tubulin(FITC). SB sample BI-IV shows extensive smooth muscle bundle formation with positive staining of critical smooth muscle cell markers. Adult samples AI-IV also show bundle formation but to a lesser degree. Cells in both samples stained positive for γ-tubulin indicating cells remained in the implanted tissue for 10 wks, were of human origin and continued to regenerate. mCherry labeled bladder SMCs that were used as a analogous controls grew to a lesser degree than both MSC populations with decreased levels of smooth muscle bundles. Due to high fluorescent intensity of cherry labeled SMCs, antibody staining could not be conducted using flurochrome conjugated antibodies due to bleed through into all channels of the fluorescent microscope. DAPI used to visualize nuclei.

FIG. 13 shows a graph of tensile stress/strain data for synthesized POC films.

FIG. 14 shows a graph of the mass of POC films versus time.

FIG. 15 shows (Panel A) a scanning electron micrograph of a POC thin film seeded with MSCs at 16 hrs; (Panel B) the in vitro MSC viability at day 1, and (Panel C) and the in vitro MSC viability at day 21 post seeding upon POC thin films. Viability was >98% at day 21 on POC thin films as quantitatively determined by live/dead staining.

FIG. 16 shows in vitro growth factor release. POC films were incubated in a 100 ng/mL of either IGF, bFGF, or VEGF solution for 3 hours at room temperature. Media was periodically removed upto 15 days. GF release was quantified using a GF specific ELISA Kit (R and D Systems, MN). Note that the release is cumulative from each semicircle scaffold (0.5 cm diameter, 0.5 mm thickness) and are statistically greater than control samples at nearly all time points

FIG. 17 shows derivation of Young's modulus for POCfs: the elastic potential of POCfs was calculated after undergoing a tensile mechanical stress evaluation, a Young's modulus of 138.53 kPa±2.85 kPa was obtained through the calculation of the slope of the tensile stress/strain data.

FIG. 18 shows degradation studies of POCfs: in vitro degradation data of POCfs through a 21 day period displayed a linear mass loss trend; by utilizing a best fit line equation, it was extrapolated that POC degrades at approximately 16 weeks.

FIG. 19 shows evaluation of POCfs: (Panel A) calcein live/dead staining of MSCs seeded onto a POCf at D1 (Panel B) same area of the same film as (a) exhibiting the massive expansion of MSCs at D21 with 98% viability, viable cells are green while non-viable cells are red, insets in (a) and (b) represent bladder SMCs and the Urotsa cell line post Calcein live/dead staining seeded onto POCfs, respectively; (Panel C) SEM of an unseeded POCf; (Panel D) MSCs that grown on the POCf for 16 hours prior to SEM analysis; (Panel E) a POCf that was typically used for augmentation procedures ; and (Panel F) MSC/UC seeded POCf immediately after being augmented into a nude rat bladder.

FIG. 20 shows Masson's trichrome staining of 4 Week POCf Augmented Tissues: (Panel A) cSMC and (Panel B) MSC outgrowth within the augmented area; and (Panel C) POCf. (Panel D-F) represent higher magnifications of (a-c), respectively. (40× magnifications).

FIG. 21 shows Masson's trichrome Staining of 10 Week POCf Augmented Tissues: (Panel A) cSMC and (Panel B) MSC outgrowth within the augmented area; and (Panel C) POCf. (D-F) represent higher magnifications of (a-c), respectively. (40× magnifications).

FIG. 22 shows Masson's Trichrome Staining of 10 Week Kidney Samples from Augmented and Control Animals. The right kidney from augmented rats was removed and stained. (Panel A) cSMC/UC POCf composite; (Panel B) MSC/UC POCf composite; (Panel C) unseeded POCf; and (Panel D) normal rat kidney. No morphological deviations were apparent from augmented and the non-augmented samples.

FIG. 23 shows quantification of trichrome staining for muscle/collagen. Both cell seeded groups showed significantly more muscle content than POCf alone, but at different time points (cSMC/UC at 4 weeks; MSC/UC at 10 weeks). Muscle content did not differ significantly between the cSMC/UC and MSC/UC groups at 4 weeks, but opposing muscle/collagen composition trends (decreased muscle for cSMC/UC and increased muscle for MSC/UC) resulted in a significant difference between the two groups at 10 weeks.

FIG. 24 shows immunofluorescent imaging of POCf/cell tissues 4 weeks post augmentation: (Panel A) cSMCs found in the augmented bladder at high levels and were dispersed throughout the augmented area only; (Panel B) MSC/POCf composite stained with human specific γ-tubulin/Alexa Red 555 antibodies; (Panel C) MSC/POCf composite stained with calponin/Alexa Red 555 antibodies and γ-tubulin/FITC antibodies; (Panel D) MSC/POCf composite stained with caldesmon/Alexa Red 555 antibodies and γ-tubulin/FITC antibodies; (Panel E) MSC/POCf composite stained with human specific elastin/Alexa Red 555 antibodies; (Panel F) MSC/POCf composite stained with γ-actin/Alexa 555 antibodies and γ-tubulin/FITC antibodies.

FIG. 25 shows immunofluorescent imaging of POCf/cell tissues 10 weeks post augmentation: (Panel A) MSC/POCf composite samples stained with human specific γ-tubulin/FITC antibodies; (Panel B) serial section of (a) stained with calponin/Alexa Red 555 antibodies and γ-tubulin/FITC antibodies; (Panel C) MSC/POCf composite stained with caldesmon/Alexa Red 555 antibodies and γ-tubulin/FITC antibodies; (Panel D) MSC/POCf composite stained with human specific elastin/Alexa Red 555 antibodies; (Panel E) MSC/POCf composite stained with human specific elastin/Alexa Red 555 antibodies; MSC/POCf composite stained with γ-actin/Alexa 555 antibodies and γ-tubulin/FITC antibodies. All samples stained with DAPI (blue) to visualize nuclei.

### DEFINITIONS

To facilitate an understanding of the present invention, a number of terms and phrases are defined below:

As used herein, the term “progenitor cell” refers to a cell that has the capacity to both proliferate, giving rise to more progenitor cells, and differentiate into one or more specific cell types or to form a specific type of tissue. “Progenitor cells” generally exhibit oligopotency, in that they are capable of differentiating into a variety of cell types. This includes uncommitted cells, preferably of mammalian origin, that are competent to differentiate into one or more specific types of differentiated cells, depending on their genomic repertoire and the tissue specificity of the permissive environment in which morphogenesis is induced. Preferably, morphogenesis culminates in the formation of differentiated tissue having structural and function properties of a tissue that occurs naturally in the body of a mammal.

The term “transplant” refers to tissue used in grafting, implanting, or transplanting, as well as the transfer of tissues from one part of the body to another, or the transfer of tissues from one individual to another, or the introduction of biocompatible materials into or onto the body. The term “transplantation” refers to the grafting of tissues from one part of the body to another part, or to another individual.

As used herein, the term “stem cell” or “undifferentiated cell” refers to self-renewing multipotent cells that are capable of giving rise to more stem cells, as well as to various types of terminally differentiated cells.

As used herein, the term “host” refers to any warm blooded mammal, including, but not limited to, humans, non-human primates, rodents, and the like, which is to be the recipient of a particular treatment. Typically, the terms “host” and “patient” are used interchangeably herein in reference to a human subject.

As used herein, the terms “defective tissues” and “defective cells” refer to tissues and cells that are marked by subnormal structure, function, or behavior. Defects responsible for the defective tissues and cells include known or detectable defects, as well as, unknown or undetectable defects.

As used herein, the term “non-human animals” refers to all non-human animals. Such non-human animals include, but are not limited to, vertebrates such as rodents, non-human primates, ovines, bovines, ruminants, lagomorphs, porcines, caprines, equines, canines, felines, ayes, etc.

The term “biologically active,” as used herein, refers to a protein or other biologically active molecules (e.g., catalytic RNA) having structural, regulatory, or biochemical functions of a naturally occurring molecule.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “isolated” when used in relation to a cell, as in “an isolated cell” or “isolated cells” refers to cells that are separated and enriched in a sample so as to remove the isolated cell(s) from other cells with which it is ordinarily associated in its natural environment. For example, isolated stem cells are stem cells that are removed from their natural environment and enriched in a sample, such that the sample housing the stem cells contains a higher percentage of stem cells than a corresponding sample found in a tissue in its natural environment.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue (e.g., tissues of the gut or central nervous system), liquid foods (e.g., milk), and solid foods (e.g., vegetables).

As used herein, “culturing” refers to propagating or nurturing a cell, collection of cells, tissue, or organ, by incubating for a period of time in an environment and under conditions which support cell viability or propagation. Culturing can include one or more of the steps of expanding and proliferating a cell, collection of cells, tissue, or organ according to the invention.

As used herein, a “recipient” refers to a mammal that receives an organ, tissue or cells taken from a donor. As used herein, a “donor” is a mammal from which organs, tissues or cells are taken for transplant into a recipient. In the case of autologous stem cells, the donor and recipient are the same subject.

### DETAILED DESCRIPTION

Developmental diseases affecting the function of the bladder have resulted in numerous attempts to create grafts for bladder replacement therapies demonstrating difficulty. The selection of proper cell types required for bladder tissue regeneration is crucial for the integrity of the replacement tissue as well as its overall function in vivo.

MSCs are multipotent cells capable of stepwise cellular transitions to terminally differentiated progeny and typically reside in a number of tissues including the BM (Pittenger et al. Science. 284:143, 1999, herein incorporated by reference in its entirety). The frequency of MSCs within the bone marrow of long bones is approximately 0.001-0.1% of nucleated cells including reticular connective tissue, monocytes/macrophages, adipocytes, and osteoblasts. Hence, MSCs are a subset of the stromal cells within the bone marrow and are not synonymous with bone marrow stromal cells in frequency and function. Clinical trial data substantiates the use of MSCs to treat hemorraghic cystitis (HC) (Ringden et al. Leukemia. 21:2271, 2007, herein incorporated by reference in its entirety). Patients receiving MSC treatment responded positively as demonstrated by a lack of detectable HC. The in vivo differentiation of human MSCs that have been genetically modified to express BMP-9 causes the induction of endochondral growth formation in an animal model without any evidence of clinical toxicity (Dayoub et al. Tissue Eng. 9:347, 2003, herein incorporated by reference in its entirety).

The growth characteristics of MSCs seeded upon a variety of scaffolds has been demonstrated. A 7-fold increase in vascular network formation is observed when utilizing EPCs and MSCs seeded in a combination upon fibrin matrices (Ghajar et al. Tissue Eng. 12:2875, 2006, herein incorporated by reference in its entirety). MSCs can not only grow on surfaces coated with collagen, but can differentiate into alternative cell lineages based upon the elastic moduli of the surface that they are seeded upon (Engler et al. Cell 126:677, 2006, herein incorporated by reference in its entirety). MSCs can proliferate on both synthetic and biologic matrices and still maintain the expression of contractile protein markers.

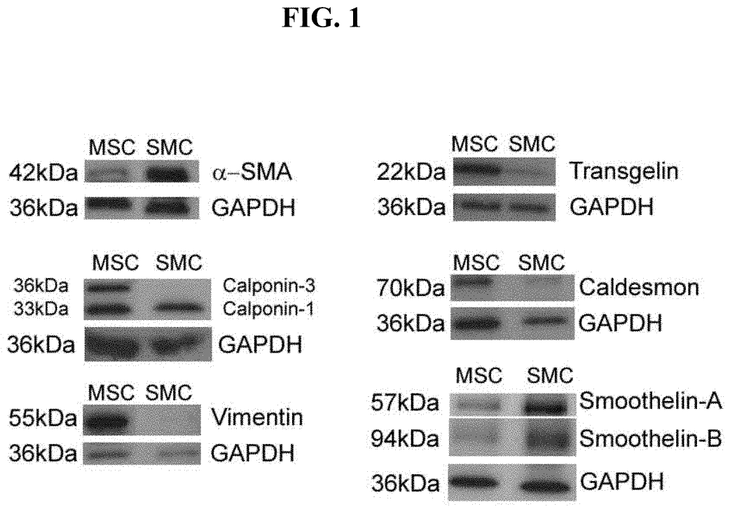

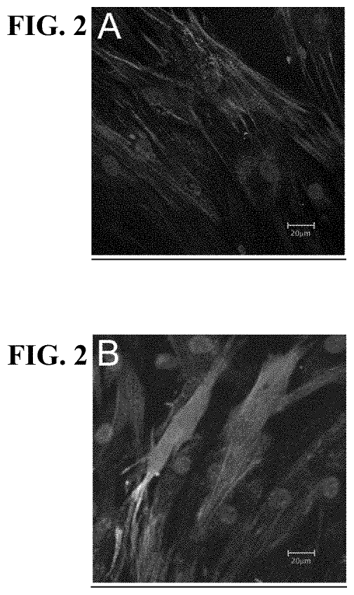

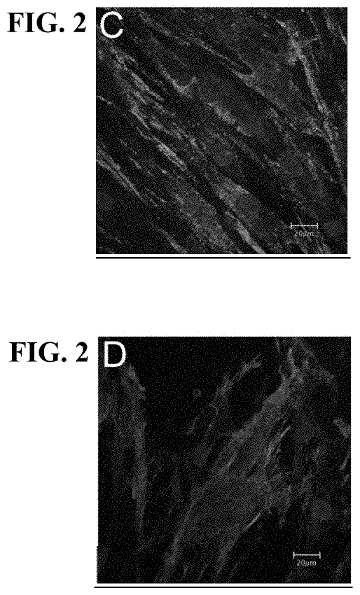

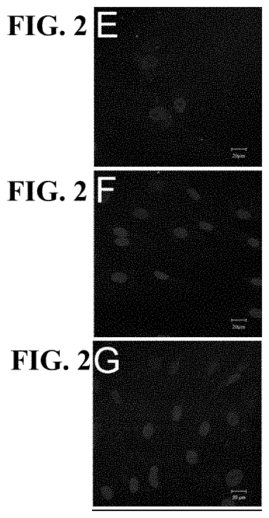

Phenotyping of cellular contractile proteins within populations of MSCs were determined by Western blot. Protein analysis revealed that the early smooth muscle marker, smooth muscle α-actin, was expressed at slightly decreased levels compared to SMCs. Expression of other contractile proteins were at levels greater than or equivalent to the SMC control sample. The calponin antibody utilized in experiments conducted during development of embodiments of the present invention recognizes three isoforms, calponin 1/2/3. Calponin 1 and 3 are expressed in MSCs while only calponin 1 is seen in SMC control (SEE FIG. 1). Vimentin, transgelin, and caldesmon are abundantly expressed as compared to the SMC sample. The antibody for smoothelin detects various isoforms of smoothelin that range from 57 to 94 kD. Both isoforms (A and B) were expressed in MSCs. Immunofluorescence staining was performed utilizing antibodies against the SMC epitopes. MSCs expressed all SMC markers with the addition of desmin and SMMHC. SMMHC is a major contractile protein whose expression exclusively marks the SMC lineage (Madsen et al. Circ Res. 82:908, 1998, herein incorporated by reference in its entirety). Overlapping of fluorochromes indicate that at least two contractile protein epitopes were present in each cell examined (SEE FIG. 2). Phenotypic data suggests that MSCs are equivalent in their expression of major contractile proteins hence MSCs may be used as an alternative to SMCs for bladder tissue regeneration. MSCs are multipotent cells; therefore, these cells also express genes of other cell types in an undifferentiated state. Hence, stimulation with cytokines would not be necessary to induce smooth muscle cell markers. However, once mobilized from the BM, local environmental cues and signaling cascades may cause the complete terminal differentiation of these cells.

The human urinary bladder encompasses an array of attributes that allow for its dynamic physiological function. Strategic goals of bladder based regenerative medicine are to develop materials that have the ability to not only mimic the native bladder milieu in the form of signaling molecules that are essential for growth but also for the development of materials that can simulate similar mechanical properties found in the bladder. Biodegradable elastic polymers have the distinct advantage of providing this function by possessing the attributes which allow them to undergo repeated expansion/contraction cycles without losing resiliency and initial mechanical fortitude making them biomechanically compatible with the organ of interest. POC [poly(1,8-octanediol-co-citrate)] is a relatively newly described synthetic polymer that has a wide range of applications including those for very elastic tissues such blood vessels as well as for orthopedic applications (Yang et al. Biomaterials 2006; 27(9):1889-1898.; Qiu et al. Biomaterials 2006;34:5845-5854.; herein incorporated by reference in their entireties). POC comprises two common compounds, citric acid and 1,8-octanediol. The elastic potential of this polymer is dependent upon several polymerization properties including the length and temperature of scaffold crosslinking which allows for modulus customization of thin films by altering the resultant polyester networks, although the present invention is not limited to any particular mechanism of action and an understanding of the mechanism of action is not necessary to practice the present invention. The elastic modulus of a human bladder is within the hundreds of kPas while typical scaffolds that are currently used for bladder tissue engineering such as non-woven PLGA, PGA, and collagen can be found in the MPa to GPa range with relatively inefficient elongation characteristics (<10%) thus making them rigid and non-contractile (Dahms et al. Br J Urol 1998;82(3):411-419.; Middleton & Tipton. Biomaterials 2000; 21(23):2335-2346.; herein incorporated by reference in their entireties). Furthermore, the degradation of these polymers typically results in a decreased localized pH causing an adverse affect upon cells in the vicinity (Kohn et al. J Biomed Mater Res 2002;60(2):292-299.; herein incorporated by reference in its entirety). It has been demonstrated that mechanical cyclical forces cause bladder smooth muscle growth and development, and static environments are less likely to produce robust contractile responses (Long-Heise et al. Tissue Eng Part A 2009 Jul. 1).

Embodiments of the present invention provide a pliable and economical thin film that can be seeded with highly characterized bone marrow MSCs as well as other bladder cell types (e.g. EPCs), and can be altered to desired specifications. In experiments conducted during development of embodiments of the present invention, POC film (POCf) was synthesized with an elastic modulus of approximately of 140 kPa and was able to elongate to 137% of its initial length without permanent deformation further demonstrating its high elastic potential. Visual inspection of the cell/POCfs immediately post-euthanization of the augmented animals revealed a film that was considerably degraded from the initial starting material at the 10 week time-point. The “patch” area consisting of the augmented bladder still exhibited pliability as well as a lack of evidence of scarification in cell seeded constructs. The cross-linking parameters at this particular elastic modulus allow for the permeability of liquid containing proteins which provide nutrients and oxygen to both faces of the scaffold resulting in a high cellular penetration depth (Kohn et al. J Biomed Mater Res 2002;60(2):292-299.; herein incorporated by reference in its entirety). In some embodiments, this attribute provides communication between the urothelium and smooth muscle components of the bladder. In some embodiments, the urothelium provides signaling elements for bladder development (Cao et al. Pediatr Res 2008;64(4):352-357.; herein incorporated by reference in its entirety).

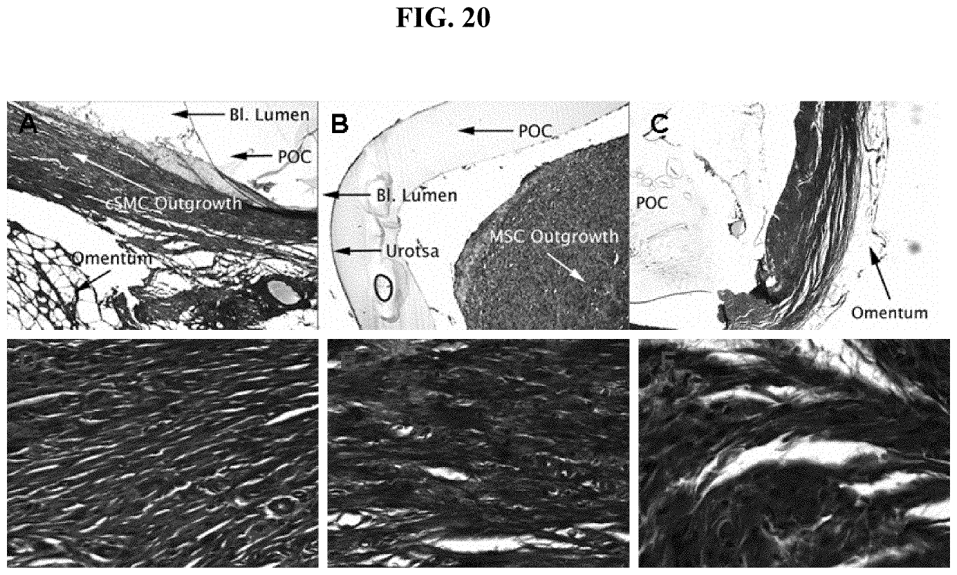

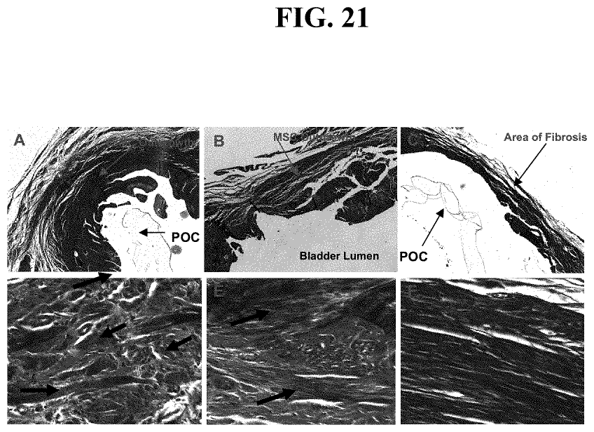

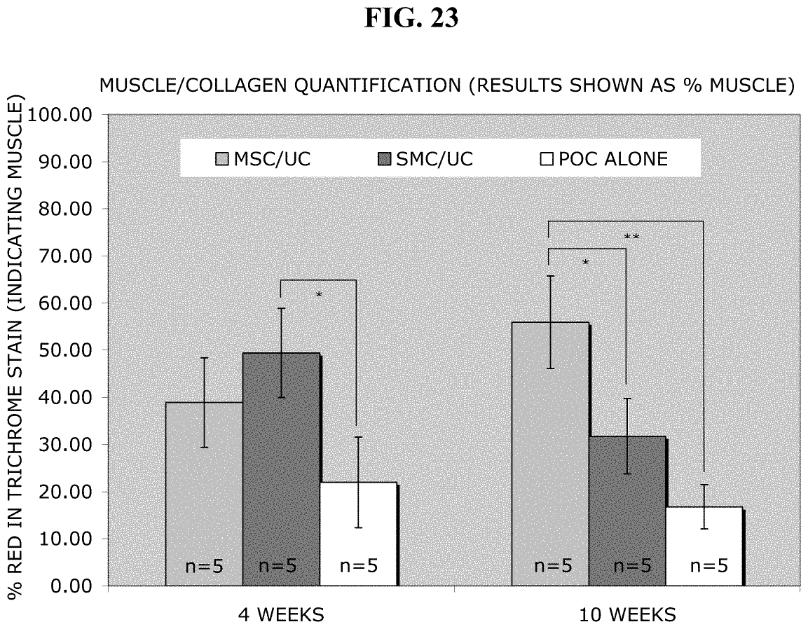

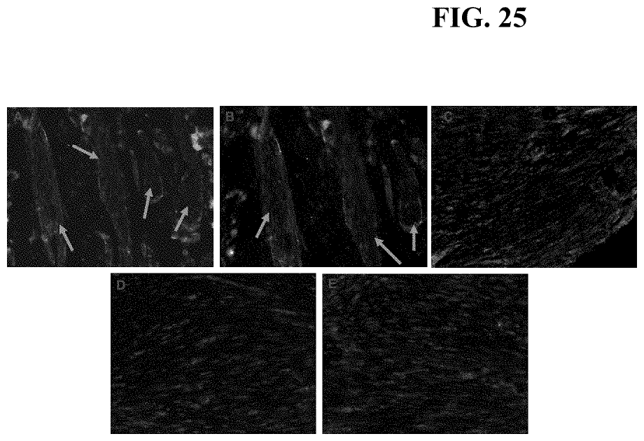

Experiments conducted during development of embodiments of the present invention tested the abilities of MSCs to act as a surrogate cell source for the regeneration of the smooth muscle component of the bladder in vivo. Bladder augmentation studies demonstrated that MSCs can assume the role of bladder SMCs and repopulate the smooth muscle layer of the bladder. The native animal bladder environment appeared to provide enough stimuli to cause the MSCs to transition from a less organized array of tissue at 4 weeks post implantation (SEE FIG. 20E) to a more defined and organized network of tissue at 10 weeks post implantation (SEE FIGS. 21E, 25A-B) as evidenced by the formation of muscle bundles that were identified as being human in origin by antibody staining. The overall architecture and thickness of the MSC based grafts were very robust and equal to or greater than those of the bladder SMC seeded control grafts indicating that the MSCs could facilitate regeneration in an area that was perceived as being damaged. Four week quantitative morphometric data demonstrated an approximate even ratio of muscle to collagen in the SMC/UC constructs but this changed dramatically at the 10 week mark where the ratio was 30%:70%. Further data demonstrated a nearly even muscle to collagen ratio of MSC/UC constructs at 10 weeks post implant which changed from approximately a 40%:60% ratio at the 4 week time-point. Unseeded POCfs remained highly fibrotic throughout the study with approximately 83% of the tissue expressing collagen at 10 weeks as a part of the natural healing process (SEE FIG. 23). Although the present invention is not limited to any particular mechanism of action and an understanding of the mechanism of action is not necessary to practice the present invention, it is contemplated that the bladder SMCs lost their ability to replicate over time due to their already terminally differentiated status, and the “stem cell” attributes of the multipotent MSCs either allowed the cells to remain quiescent until recruitment by environmental cues or they were committed towards a path of differentiation and continued to robustly grow within the graft. In some embodiments, it is of vital clinical relevance when isolating MSCs from a patient population to be able do so in a highly reproducible manner; hence, the use of epitope defined cells, without also acquiring contaminating cells that are non-exclusively present within the bone marrow including adipocytes, osteocytes, dendritic cells, and macrophages that may be inhibitory to the regenerative process (Lichtman et al. Williams Hematology 7th Edition. New York: McGraw-Hill Professional, 2005. p. 35-72.; herein incorporated by reference in its entirety). Experiments conducted during development of embodiments of the present invention demonstrate that populations of multipotent, bone marrow derived human MSCs can be utilized in a bladder augmentation model to provide smooth muscle cell regeneration.

Immune generated inflammatory responses to implanted foreign objects manifest themselves in the form of localized scarring in which high levels of collagen are present (Hu et al. Blood. 2001;98(4):1231-1238.; herein incorporated by reference in its entirety). Initial SMC/UC constructs demonstrated a near normal muscle collagen ratio at 4 weeks, indicating that the increased levels of collagen at the 10 week time-point was due to the natural life cycle of the bladder SMCs and not created due to a localized immune response by the host towards the POCf construct or an immunosuppressive effect by the seeded bladder SMCs since these cells are not known to possess any immunosuppressive attributes. Furthermore, MSC/UC constructs did not display an increase in collagen content throughout the study nor a fibrotic encapsulation of the construct, suggesting that POCfs do not elicit an immune response which is further corroborated by studies in immunocompetent animals (Qiu et al. Biomaterials 2006;34:5845-5854.; Kibbe et al. J Biomed Mater Res A. 2010 ;93(1):314-324.; herein incorporated by reference in their entireties).

The formation of calculi is commonly composed of calcium or magnesium based stones. These potentially obstructive elements manifest themselves in the forms of calcium oxalate, calcium phosphate, or magnesium phosphate and may be found within the bladder of augmented nude rats (Piechota et al. Urol Res 1999;27(3):206-213.; herein incorporated by reference in its entirety). Depending upon the locale and degree of formation, the calculi may interrupt basic physiological processes in the bladder. Animals that have been augmented with biological materials such as pro-inflammatory SIS in a cell seeded or unseeded manner, or sham controls had an approximate 50% rate of calculi formation throughout the luminal side of the bladder ranging in size, shape and hardness. There are a variety of explanations as to why calculi formation may occur in vivo, but it is believed that underlying metabolic conditions or calcium and/or magnesium rich diets may play a crucial role since calculi that have been examined contain high levels of these chemical elements (Rizvi et al. J Urol 2002;168(4 Pt 1):1522-1525.; herein incorporated by reference in its entirety). POCfs, utilized throughout studies conducted during development of the present invention, exhibited a very low occurrence of calculi formation (e.g. 12% in all augmented animals). Although the present invention is not limited to any particular mechanism of action and an understanding of the mechanism of action is not necessary to practice the present invention, the low level of calculi formation may be due to the leaching of citrate from the degrading POCfs. Citrate is a known chelator of divalent cations such as calcium and magnesium and is often used to prevent stone formation in surgical patients by competing with oxalate for free calcium (Robinson et al. J Urol 2009;181(3):1145-1150.; herein incorporated by reference in its entirety). Experiments conducted during development of embodiments of the present invention indicate that POC provides scaffolds for bladder regeneration which lessen the incidence of calculi formation.

The MSCs utilized in experiments conducted during development of embodiments of the present invention retained known markers of bladder smooth muscle physiology at 10 weeks post implant and were rigorously tested with antibodies specific for smooth muscle γ-actin, α-SM, calponin, caldesmon, and elastin. γ-actin was specifically chosen for testing since it is known for its expression on differentiated bladder SMCs (Baskin et al. J Urol 1996;156(5):1820-1827.; herein incorporated by reference in its entirety). Data indicate that smooth muscle epitopes were present in augmented bladders and co-localized with a human specific γ-tubulin protein indicating that the cells in the graft were of human origin. Recent studies have demonstrated the utility of using mechanical stimulation to promote the upregulation of the protein elastin in bladder SMCs (Long-Heise et al. Tissue Eng Part A 2009 Jul. 1; herein incorporated by reference in its entirety). Elastin is a key component in bladder SMC physiology and allows the bladder to contract and expand after being subjected to deformation. Decreased elastin expression leads to poor bladder mechanical properties and subsequently affects the function of the bladder at numerous levels as demonstrated by the non-compliant bladder (Djavan et al. J Urol 1998;160(5):1658-1662.; herein incorporated by reference in its entirety). MSCs express low levels of elastin in two dimensional cultures in vitro but are upregulated in three dimensional settings in vivo as demonstrated by human specific elastin antibody staining of our MSC/POCfs augmented bladders. It is contemplated that MSCs seeded within the scaffold act as a beacon to recruit MSCs from a variety of tissue sources that include the bone marrow as well as adipose tissue that may be found within close proximity to the bladder.

Experiments were conducted during development of embodiments of the present invention to determine whether each cell population would also contract and proliferate equivalently in vitro. A hallmark characteristic of neurogenic bladder is a lack or decrease in coordinated bladder contractility (Lin et al. J Urol. 171:1348, 2004, herein incorporated by reference in its entirety). The in vitro contractile potential of BMSPCs was evaluated via stimulation with carbachol. No difference was observed with regard to magnitude of response. Cellular proliferative capacity was examined to determine differences in growth relevant to the use of MSCs in a bladder regeneration model. Contractile and proliferative responses were statistically indistinguishable from SMCs suggesting that both cell populations may be used interchangeably in an in vivo setting.

The developmental origins of EPCs and eventual usage in tissue regeneration have been demonstrated (Ghajar et al. Tissue Eng. 12:2875, 2006, Lin et al. J Urol. 171:1348, 2004, Pusztaszeri et al. J Histochem Cytochem. 54:385, 2006, Fang et al. Chin Med J (Engl). 120:696, 2007, herein incorporated by reference in their entireties). In experiments in which decellularized porcine aortic heart valves were seeded with EPCs resulting in complete endothelialization of the valves, EPCs were tested to demonstrate that relevant populations of vasculature producing EPCs could be obtained and differentiated along the endothelial lineage. Coerced differentiation of EPCs phenotypically demonstrated the ability of EPCs to transform form immature EPCs into more mature endothelial cells through the acquisition terminally differentiated endothelial markers. In vivo analysis of EPC seeded CAM clearly demonstrated that vasculature formation is possible with human derived cells.

In some embodiments, the present invention provides compositions and methods for tissue regeneration (e.g. organ tissue (e.g. bladder tissue, etc.). In some embodiments, the present invention provides cells and other materials (e.g. scaffolds) for transplantation and/or regeneration of cells, tissues, and/or organs. In some embodiments, transplantation and/or regeneration occur in vitro or in vivo. In some embodiments, provides autologous cells (e.g. MSCs, EPCs, etc.). In some embodiments, provides epitope-defined cells (e.g. MSCs, EPCs, etc.). In some embodiments, provides multipotent cells (e.g. MSCs, EPCs, etc.). In some embodiments, provides bone marrow derived cells (e.g. MSCs, EPCs, etc.). In some embodiments, the present invention provides the autologous sources of epitope defined, multipotential, bone marrow derived mesenchymal stem and endothelial progenitor cells (EPCs) for bladder regeneration. Mesenchymal stem cells (MSCs) have the ability to differentiate into a variety of cell types including adipocytes, osteocytes, chondrocytes, neural cells, and muscle cells. EPCs have the ability to undergo angiogenesis and promote vascularization of newly developing tissue. In some embodiments, both MSCs and EPCs can be harvested from the bone marrow simultaneously. MSCs and EPCs are unaffected by trauma or disease states. In some embodiments, MSCs and EPCs are seeded into biological or synthetic scaffolds and surgically implanted into animal models or patients to contribute to bladder regeneration. In some embodiments, the desired cells (e.g. cell mixture, MSCs, and/or EPCs) are obtained, isolated, and/or purified by any suitable techniques known to those of skill in the art. In some embodiments, the desired cells (e.g. cell mixture, MSCs, and/or EPCs) are obtained, isolated, and/or purified by techniques described herein.

The present invention is not limited by the type of scaffold. In some embodiments, the scaffold comprises a web, matrix, and or thin film. In some embodiments, a matrix comprises one or more biodegradable elastomers. In some embodiments, a matrix comprises one or more poly(diol citrates) (e.g. Poly(1,8 octanediol-co-citrate) (POC), poly(1,6-hexanediol-co-citrate) (PHC), poly(1,10-decanediol-co-citrate) (PDC), poly(1,12-dodecanediol-co-citrate) (PDDC), poly(1,8-octanediol-co-citrate-co-MDEA) (POCM 10%), poly(1,12-dodecanediol-co-citrate-co-MDEA) (PDDCM 10%), etc.). In some embodiments, the present invention provides a scaffold comprising POC. In some embodiments, POC is configured to form a thin film scaffold. In some embodiments, POC provides a flexible, biodegradable, non-toxic, and/or sutural thin-film scaffold. In some embodiments, POC exhibits the capacity to function as a useful scaffold in both in vivo and in vitro settings. In some embodiments, a POC scaffold is transplantable with a desired cell mixture (e.g. MCSs and EPCs). In some embodiments, a scaffold (e.g. POC scaffold) provides a substrate upon which to transplant a desired cell mixture (e.g. MCSs and EPCs). In some embodiments, a scaffold (e.g. POC scaffold) provides a growth surface and/or material for a desired cell mixture (e.g. MCSs and EPCs) upon transplantation. In some embodiments, a scaffold (e.g. POC scaffold) is configured to remain as part of new tissue (e.g. bladder tissue) following transplant. In some embodiments, a scaffold (e.g. POC scaffold) is configured to remain associated with transplanted cells (e.g. MCSs and EPCs) and/or regenerated tissue (e.g. bladder tissue). In some embodiments, a scaffold (e.g. POC scaffold) is configured to degrade following transplantation (e.g. hours after transplantation, days after transplantation, weeks after transplantation, months after transplantation, years after transplantation, etc.). In some embodiments, a scaffold (e.g. POC scaffold) is configured to degrade following tissue regeneration (e.g. hours after transplantation, days after transplantation, weeks after transplantation, months after transplantation, years after transplantation, etc.).

In some embodiments, MSCs and EPCs express key contractile proteins that are a requirement for overall bladder function. In some embodiments, MSCs allow for urinary bladder regeneration. In some embodiments, MSCs can replace the muscle component of the urinary bladder wall. In some embodiments, the muscle component of the urinary bladder wall is defective in patients with developmental or acquired trauma or disease. In some embodiments, MSCs are used to overcome the shortcomings of bladder regenerative medicine. In some embodiments, MSCs and/or EPCs are derived from the species in which they are transplanted. In some embodiments, MSCs and/or EPCs are derived from a mammalian source (e.g. human, non-human primate, canine, feline, rodent, etc.). In some embodiments, MSCs and/or EPCs are derived from a human. In some embodiments, MSCs and/or EPCs are derived from autologous sources. In some embodiments, the use of autologous sources of bone marrow derived MSCs eliminates the danger of immune response, long term immunosuppressive therapy, and post-implantation immunorejection.

In some embodiments, the present invention provides isolated stem cells (e.g. as a treatment or therapy). In some embodiments, the present invention provides isolated messenchymal stem cells (MSCs). In some embodiments, the present invention provides isolated endothelial progenitor cells (EPCs). In some embodiments, the present invention provides purified stem cells (e.g. MSCs and/or EPCs). In some embodiments, the present invention provides isolated and/or purified stem cells (e.g. MSCs and/or EPCs) for regenerative medicine (e.g. tissue regeneration, organ regeneration, bladder regeneration). In some embodiments, the present invention provides isolated MSCs and/or EPCs for bladder regeneration. In some embodiments, MSCs and/or EPCs are isolated from one or more other cell types. In some embodiments, MSCs and/or EPCs are provided with one or more additional cell types (e.g. other cells isolated from bone marrow). In some embodiments, MSCs and/or EPCs are substantially isolated from other cells, however, some residual cells or cell types remain with the MSCs and/or EPCs. In some embodiments, MSCs and/or EPCs are purified from contaminants. MSCs and/or EPCs are substantially purified from contaminants.

In some embodiments, the present invention provides an in vitro cell culture. In some embodiments, the stem cells are self-renewable (e.g., in vitro or in vivo). In some embodiments, the stem cells of the present invention are multipotent.

In some embodiments, the present invention provides methods for using the stem cells (e.g. MSCs, EPCs, etc.). In some embodiments, the present invention provides a method for transplanting stem cells in vivo, for example, comprising the steps of 1) providing: a host organism and isolated stem cells (e.g. MSCs, EPCs, etc.); and transplanting the stem cells into a tissue of the host. In some preferred embodiments, the stem cells are transplanted into an injured tissue of the host. In particularly preferred embodiments, the stem cells that are transplanted into the host are also derived from the host (i.e., auto-transplantation).

In some embodiments, the present invention provides methods for enhancing the ability of endogenous stem cells to provide therapeutic benefits to the surrounding tissue (e.g., injured tissue), comprising the step of administering to a host having stem cells (e.g. MSCs and/or EPCs) an effective dose of a compound that promotes proliferation of differentiation of the stem cells (e.g. MSCs and/or EPCs), thereby therapeutically augmenting the ability of the stem cell to promote repair after injury. In some embodiments, the compound is a compound that directly or indirectly provides a growth factor to the stem cells (e.g., EGF, FGF).

In some embodiments, a stem cell according to the invention is immunologically blinded or immunoprivileged. As used herein, “immunologically blinded” or “immunoprivileged” refers to a cell that does not elicit an immune response. As used herein, an “immune response” refers to a response made by the immune system to a foreign substance. An immune response, as used herein, includes but is not limited to transplant or graft rejection, antibody production, inflammation, and the response of antigen specific lymphocytes to antigen. An immune response is detected, for example, by determining if transplanted material has been successfully engrafted or rejected, according to methods well-known in the art. In some embodiments, an “immunogically blinded stem cell” or an “immunoprivileged stem cell” according to the invention can be allografted or xenografted without transplant rejection, and is recognized as self in the transplant recipient or host.

In some embodiments “isolating” a stem cell refers to the process of removing a stem cell from a tissue sample and separating away other cells which are not stem cells of the tissue. An isolated stem cell will be generally free from contamination by other cell types and will generally have the capability of propagation and differentiation to produce mature cells of the tissue from which it was isolated. However, when dealing with a collection of stem cells, e.g., a culture of stem cells, it is understood that it is practically impossible to obtain a collection of stem cells which is 100% pure. Therefore, an isolated stem cell can exist in the presence of a small fraction of other cell types which do not interfere with the utilization of the stem cell for analysis or production of other, differentiated cell types. Isolated stem cells will generally be at least 30%, 40%, 50%, 60%, 70%, 80%, 85%, 90%, 95%, 98%, or 99% pure. Preferably, isolated stem cells according to the invention will be at least 98% or at least 99% pure.

A stem cell, progenitor cell, or differentiated cell is “transplanted” or “introduced” into a mammal (e.g. human or non-human subject) when it is transferred from a culture vessel into a patient. Transplantation, as used herein, can include the steps of isolating a stem cell according to the invention and transferring the stem cell into a mammal or a patient. Transplantation can involve transferring a stem cell into a mammal or a patient by injection of a cell suspension into the mammal or patient, surgical implantation of a cell mass into a tissue or organ of the mammal or patient, or perfusion of a tissue or organ with a cell suspension. The route of transferring the stem cell or transplantation will be determined by the need for the cell to reside in a particular tissue or organ and by the ability of the cell to find and be retained by the desired target tissue or organ. In the case where a transplanted cell is to reside in a particular location, it can be surgically placed into a tissue or organ or simply injected into the bloodstream if the cell has the capability to migrate to the desired target organ.

Transplantation, as used herein, can include the steps of isolating a stem cell according to the invention, and culturing and transferring the stem cell into a mammal or a patient. Transplantation, as used herein, can include the steps of isolating a stem cell according to the invention, differentiating the stem cell, and transferring the stem cell into a mammal or a patient. Transplantation, as used herein, can include the steps of isolating a stem cell according to the invention, differentiating and expanding the stem cell and transferring the stem cell into a mammal or a patient.

The invention also provides for a pharmaceutical composition comprising the isolated stem cells of the invention admixed with a physiologically compatible carrier.

### EXPERIMENTAL

### Example 1

### Compositions and Methods

### BMSPCs Characterization.

MSCs from passage 5 or earlier were used (1-2 weeks in culture) and grown in Mesenchymal Stem Cell Growth Media (Lonza) to support maintenance but not the differentiation of MSCs. Sub-populations of human BM CD34+/CD133+ (termed EPCs) were isolated via FACS from CD34+ cells (Lonza) using 250 ng of CD34 PerCP-Cy5.5 (BD Pharmingen, San Jose Calif.) and a 1:100 dilution of CD133-PE (Miltenyi Biotec, Auburn, Calif.) antibodies. EPCs were maintained in M199 with 5% FCS and stimulated with vascular endothelial growth factor (VEGF, 50 ng/ml), basic fibroblast growth factor (bFGF, 10 ng/ml), and insulin like growth factor-1 (IGF-1, 10 ng/ml) for 14 days. All cells were grown at 37° C. in a humidified incubator with 5% CO₂ in air. Growth factors were purchased from R & D Systems, Minneapolis, Minn.. Morphologic evaluation of MSCs did not demonstrate spontaneous differentiation into adipocytes or osteocytes during cell culturing. Each lot of MSCs was tested for purity and their ability to undergo multilineage differentiation.

### Immunofluorescent Imaging

Immunofluorescent imaging performed on MSCs grown on Lab-Tek chamber slides (Nunc, Rochester N.Y.) were fixed/permeabilized utilizing the Intracellular FCM System (SCBT, Santa Cruz, Calif.). MSCs were stained with antibodies (SCBT) against desmin, smooth muscle myosin heavy chain (SMMHC), smooth muscle α-actin (SMA), calponin, caldesmon, vimentin, transgelin, and smoothelin (1:100 to 1:200 dilution). Fluorescein isothiocyanate (FITC) or Alexa Red 555 (Invitrogen, Carlsbad, Calif.) conjugated secondary antibodies (used at 1:200) were used as detection reagents for primary antibodies lacking directly conjugated fluorochromes.

### Western Blotting

20 μg of total protein was harvested from MSCs and SMCs utilizing standard procedures. Western blot analysis was used to detect SMA, calponin, vimentin, transgelin, smoothelin, and caldesmon. Levels of glyceraldehyde-3-phosphate-dehydrogenase (GAPDH) were also evaluated to assess protein integrity.

### Cell Proliferation Assay

MSCs or SMCs were seeded into 96-well plates at 3K cells/well followed by 12 hours of serum deprivation. Cells were then grown in 0.5% FCS containing MSC media or 10% FCS containing SMC media for up to 15 days. At days 1, 3, 7, 11 and 15 media was removed and the Cyquant Cell Proliferation Assay (Invitrogen) was performed. Each plate included MSC and SMC standards for use in converting fluorescence intensity values into cell numbers. For each time point, a mean cell number was calculated.

### Intracellular Calcium Release Assay

MSCs or SMCs were seeded in 96-well plates at 20K cells/well and grown overnight in MSC or SMC media. The Fluo-4 Calcium Assay (Invitrogen) was utilized to determine Ca2+ flux as a surrogate assay for contractility. Cells were treated with a 10 mM carbachol (Sigma, St. Louis, Mo.) solution. Fluorescence values were recorded pre- and post-stimulation, and background fluorescence subtracted out. Cells were also visualized using confocal microscopy.

### Chorioallantoic Membrane (CAM) Model

Fertilized chicken eggs were utilized as described. GFP+EPCs were seeded into the CAM and incubated until day 18. Samples were then processed as described. Sections were subjected to a human specific major histocompatibility complex I (MHC I,) (SCBT; 1:100 dilution; no cross-reactivity with chicken cells) antibody staining and secondary staining achieved with Histostain Plus (Invitrogen).

POC [poly(1,8-octanediol-co-citrate)] Thin Film Synthesis and Mechanical Properties Characterization

In some embodiments, POCf synthesis comprised the addition of equimolar amounts of citric acid and 1,8 octanediol (Sigma Aldrich, St. Louis, MO) which were combined and melted at 160-165° C. with stirring (Atala et al. J Urol 1993;150(2 Pt 2):608-612.; Yang et al. Biomaterials 2006; 27(9):1889-1898.; herein incorporated by reference in their entireties). Following melting, the temperature of the solution was reduced to 140° C. for 25 minutes and cooled to create a pre-polymer. The pre-polymer was then dissolved in ethanol to produce a 30% w/v solution and poured into an untreated flat bottomed glass mold. The mold was transferred to an oven and post-polymerized for 7 days at 55° C. Following post-polymerization, POC films were de-molded and unreacted monomer was removed by incubation in 1× DMEM (Lonza Inc. Walkersville, Md.) media with media changes every 6 hours for 24 hours. Tensile mechanical tests were performed with an Instron 5544 mechanical tester (Instron, Grove City, Pa.) equipped with a 500 N load cell. 5.0 cm×1.0 cm POCf samples were pulled at a rate of 500 mm/min. Young's modulus was calculated from the slope of the stress/strain line. Degradation studies of POCfs were performed as follows: POCfs were prepared as previously described and swelled by standard methods. Uniform POC circular disks (diameter 0.5 cm, thickness 0.5 mm) were cut and lyophilized for 3 days. Following lyophilization, POC disks were weighed and then subsequently rehydrated in PBS at 37° C. in an incubator with 5% CO₂ in air. Individual POC disks of known mass were placed in 0.75 mL MSC growth media (Lonza Inc.) and degraded at 37° C. with 5% CO₂ in air. Media was changed on a weekly basis and POC disks were removed semiweekly, rinsed in PBS, lyophilized, and subsequently weighed. Percent mass loss was measured by the following equation: % Mass Loss=(Mₒ−M)/Mo*100% where Mo is initial mass of the film and M is mass lost of the POC. POCfs did not exhibit autofluorescent characteristics when observed under fluorescent light between the wavelengths of 488-700 nm.

### Transduction of Bladder SMCs

The self-inactivating (SIN) lentiviral vector, pCE-EF1αGFP, was used to create lentivirus (LV) pseudotyped with the vesicular stomatitis virus G (VSV-G) envelope protein (Cui et al. Blood 2002, 99(2):399-408.; herein incorporated by reference in its entirety). The SIN vector allows for the production of replication incompetent virus so that transduced populations of cells cannot produce virus. A secondary construct was created in which the mCherry Fluorescent Protein (CFP) was exchanged for the GFP in order to label SMCs for in vivo tracking and aid in determining the origin of the cells in regenerated tissue. The resulting plasmid was pCE-EF1αCFP. Recombinant lentivirus was generated using a three plasmid based system by co-transfection of 293T cells (ATCC, Manassas, Va.) through calcium phosphate precipitation (Cui et al. Blood 2002, 99(2):399-408.; herein incorporated by reference in its entirety). In addition to the transducing vector pCE-EF1αCFP, two other vectors were used in viral production. pMD.G, which expresses the VSV-G envelope protein and pCMVAR8.91, which expresses the HIV-1 gag/pol, tat, and rev genes are both required for efficient LV production. The ratio of DNA of the transducing vector pCE-EF1αCFP, pMD.G, and pCMVAR8.91 was 1.5:0.5:2 μg for 10⁶ 293T cells for transfection when plated in a 35-mm dish. Supernatant containing virus were harvested at 72 hours post transfection and passed through a 0.4 micron filter which separated virus from transfected 293T virus producing cells. Filtered supernatant was co-cultured with normal human bladder SMCs (Lonza Inc.) for two days in a humidified tissue culture incubator at 37° C. with 5% CO₂ in air resulting in CFP labeled SMCs (cSMCs). Viral titers were obtained (Cui et al. Blood 2002, 99(2):399-408.; herein incorporated by reference in its entirety) and generally ranged from 0.5-5×10⁶ viral particles/ml.

In vitro Cell Viability

Viability of epitope defined human bone marrow derived, CD29⁺CD44⁺CD105⁺CD166⁺ MSCs (Lonza Inc.) seeded upon POCfs was determined in vitro 21 days post-seeding via a Calcein Live/Dead Cytotoxicity Kit (Molecular Probes, Carlsbad, Calif.) according to manufacturer's instructions. MSCs are tested for their multipotentiality via standard cell based assays by the supplier. MSC seeded POCfs (5,000 cells/cm²; 0.50 cm×0.75 cm) grown in MSC media (Lonza Inc.) were washed with 3 volume exchanges (20 mls) of Dulbecco's Phosphate Buffered Saline (DPBS; Lonza Inc.) to remove any endogenous esterase activity typically found in serum containing media. 25 ul of the 2 mM EthD-1 (ethidium homodimer) stock solution was combined with 10 ml of sterile DPBS and 12.5 ul of the 4 mM calcein AM stock solution was subsequently added to this solution resulting in a 5 uM calcein AM and 5 uM EthD-1 working solution. MSC/POCfs were incubated in the solution for 15 minutes at 37° C. followed by imaging with an epifluorescent microscope and quantified.

### Cell Seeding Studies on POCfs

POCfs (0.50 cm×0.75 cm) that were utilized for bladder augmentation studies were seeded accordingly: MSCs or cSMCs were seeded at 15,000 viable cells/cm² in conjunction with the well described urothelial cell (UC) line, Urotsa (Rossi et al. Environ Health Perspect 2001;109(8):801-808.; herein incorporated by reference in its entirety), on opposing sides of the POCf in the cSMC/UC or MSC/UC composites. MSCs or cSMCs were seeded for 4 hours on a side of the POCf in either MSC or SMC media respectively (Lonza Inc.) and then flipped over and seeded with 20,000 viable cells/cm2 of the Urotsa cell line. These composites were cultured for 9-11 days in a humidified cell incubator at 37° C. with 5% CO₂ in air prior to their use in augmentation procedures. MSCs or SMCs were used at passage 5 or earlier.

### Nude Rat Bladder Augmentation Utilizing Cell/POCf Composites

Adult athymic female nude rats (NCI Animal Production Program, Frederick, Md.) weighing approximately 200-250 g underwent bladder augmentation cystoplasty (Aslan et al. Urol Res 2004; 32(4):298-303.; herein incorporated by reference in its entirety). Animals were anesthetized with 60 mg/kg ketamine and 5 mg/kg xylazine intraperitoneally (IP) and given an analgesic injection of Buprenex (1 mg/kg) administered subcutaneously (SQ) to decrease any pain during and after surgery. A 1 cm midline vertical abdominal incision was created to expose the abdominal wall and physical separation of the abdominal wall led to the identification of the bladder. A 40-50% supratrigonal cystectomy was performed from anterior to posterior creating a clamshell. The cystectomized defect was augmented with the aforementioned cell/POCf composites: 1) cSMC/UC POCf; 2) MSC/UC POCf; 3) unseeded POCf alone (negative control). The bladder was then closed with 7-0 polyglactin suture in a water-tight fashion and was enveloped with omentum in the area containing the POCf or POCf/cell composites. Permanent marking sutures of 6.0 nylon were placed at the 12, 3, 6, and 9:00 positions on the outside of the bladder for identification purposes at the time of sacrifice. The abdominal wall was then closed in a single layer with 5-0 ethibond running suture and the skin re-approximated with 9 mm autoclips. Each group was sacrificed at 4 and 10 week time-points. All pre- and post-animal procedures were performed in accordance with guidelines set forth and approved by the Children's Memorial Hospital Institutional Animal Care and Use Committee (IACUC).

### Trichrome Staining of Augmented POCf Tissue Composites

Full thickness bladder tissue specimens were isolated immediately following euthanasia of animals and were fixed in 10% buffered formalin phosphate (Fisher Scientific, Pittsburgh, Pa.) and dehydrated through a series of graded ethanol exchanges then embedded in paraffin according to well established protocols at 4 and 10 week time-points (Lin et al. J Urol 2004;171(3):1348-1352.; Beqaj et al. J Urol 2005; 174(4 Pt 2):1699-1703.; herein incorporated by reference in their entireties). Embedded tissues were sectioned at a thickness of 5-15 μm using a RM2125 RT Microtome (Leica, Nannockburn, Ill.) onto glass slides and subjected to Masson's Trichrome (Sigma-Aldrich) staining (Beqaj et al. J Urol 2005; 174(4 Pt 2):1699-1703.; herein incorporated by reference in its entirety). Tissue containing slides were deparaffinized by incubating slides at 62° C. for 7 min on a hot plate followed by treatment with xylenes, graded ethanol washes and deionized water. Slides were placed in Bouin's solution for 15 min then rinsed under running tap water. Hematoxylin staining for 5 min followed Bouin's staining and was followed by rinsing and 5 minutes of staining with Scarlet-Acid Fuchsin. Following a rinse with deionized water, slides were subjected to a mixture of PTA/PMA, followed by Aniline Blue solution and a 1% acetic acid wash. Lastly, slides were placed in 95-100% ethanol and rinsed in xylene. After drying, a coverslip was placed over the tissue and secured with 3-4 drops of Permaslip (Alban Scientific Inc, St Louis, Mo.). Kidney samples were also removed from each animal and similarly subjected to the aforementioned preparations and Masson's Trichrome staining to determine whether there were any detrimental effects upon the kidney structure based upon gross morphology of the newly augmented bladders.

### Quantification of Trichrome Stained POCf Composites

Explanted bladder tissue containing of cell/POCf composites that underwent the aforementioned Trichrome staining were evaluated for muscle and collagen content (Slaughenhoupt et al. J Urol 1999;162(6):2119-2122.; herein incorporated by reference in its entirety). Muscle to collagen fiber ratio from Trichrome stained samples was quantified digitally utilizing a Nikon Eclipse 50i Microscope (Nikon Inc., Melville, N.Y.) and Spot Advanced Imaging Software (Diagnostic Instruments, Sterling Heights, Mich.). Sample images (1600 pixels×1200 pixels, bit depth 24) were opened with Adobe Photoshop CS3 (Adobe Systems Inc., San Jose, Calif.). The contrast of red pixels from blue pixels was enhanced by a two-fold elevation of magenta levels followed by a two-fold depression of cyan levels in the red and magenta spectra. This contrast was further improved by a two-fold elevation of cyan levels followed by a two-fold depression of magenta levels in the cyan and blue spectra. The selection color range tool with a fuzziness level of 115% was then used to digitally select the red or blue pixels of the entire image. Selected pixels were subsequently quantified using the image histogram tool and a muscle to collagen ratio was calculated from these values. In cases where urothelial cells, red blood cells, or debris were present, images were edited to remove these structures to preserve a more accurate extrapolation of the muscle:collagen from the red:blue.

### Immunofluorescent Characterization of Augmented Tissues

Following the dehydration, embedding and depariffinization process, sectioned tissue samples were also subjected to immunofluorescent staining. Slides were subjected to antigen retrieval consisting of 15 min of boiling in citrate buffer (0.01M citrate solution, pH 6.0 with 0.05% Tween-20) and then cooled to room temperature. Staining consisted of a blocking step for 15 min in bovine serum albumin (BSA, 5 mg/ml) followed by a 40 min incubation at room temperature with the primary antibody. After washing with DPBS, slides were incubated for 30 min with a secondary antibody and eventually rinsed with DPBS and air dried. Slides were mounted with Vectashield (Vector Laboratories, Burlingame, Calif.). Primary antibodies were directed against epitopes for markers of bladder smooth muscle cells utilizing antibodies against smooth muscle α-actin, smooth muscle γ-actin, calponin, caldesmon and human specifc γ-tubulin and elastin (Santa Cruz Biotech, Santa Cruz, Calif.) in conjunction with either an Alexa Red 555 or FITC conjugated secondary antibodies (Molecular Probes, Carlsbad, Calif.) following established protocols. Primary antibodies were utilized at dilutions between 1:100 to 1:250 while secondary antibodies were utilized at a 1:400 dilution. All samples were additionally stained with 4′,6-diamidino-2-phenylindole (DAPI) to identify cells by nuclei visualization.

### Example 2

### Bladder Regeneration by MSCs and EPCs

### MSC Phenotyping

MSCs were grown in MSC media supplemented with 0.5% FBS to assess the basal levels of contractile protein markers known to be present in SMCs. A diminished level of serum was utilized to eliminate erroneous modulation of protein expression caused by cytokines known to be found in serum. Western blot data demonstrated endogenous levels of key SMC contractile proteins including human SMA; smoothelin; transgelin; caldesmon; calponin; and vimentin present in MSCs with varying degrees of expression (SEE FIG. 1). Antibody staining for GAPDH demonstrated that the isolated protein retained its structural integrity. Immunofluorescence data corroborated Western blot analyses which showed that MSCs expressed SMC markers (SEE FIG. 2). Two additional contractile protein markers, SMMHC and desmin (SEE FIGS. 2A and 2C) were also examined and demonstrate positive staining for these markers on MSCs. These analyses suggest that MSCs possess similar intracellular structural elements commonly found in SMCs required for cell contractility.

### Cell Proliferation Assay

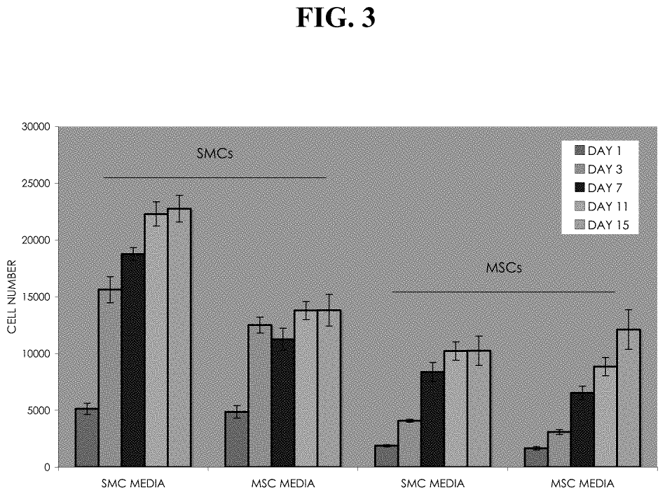

During the early to mid-phase of the proliferation assay (days 1-7), SMCs and MSCs grown in SMC media demonstrated a greater proliferative capacity versus mid to late (days 8-15) time-points. For both cell types, 77% of the total (days 1-15) growth occurred by day 7. (SEE FIG. 3).

### Intracellular Calcium Release Assay

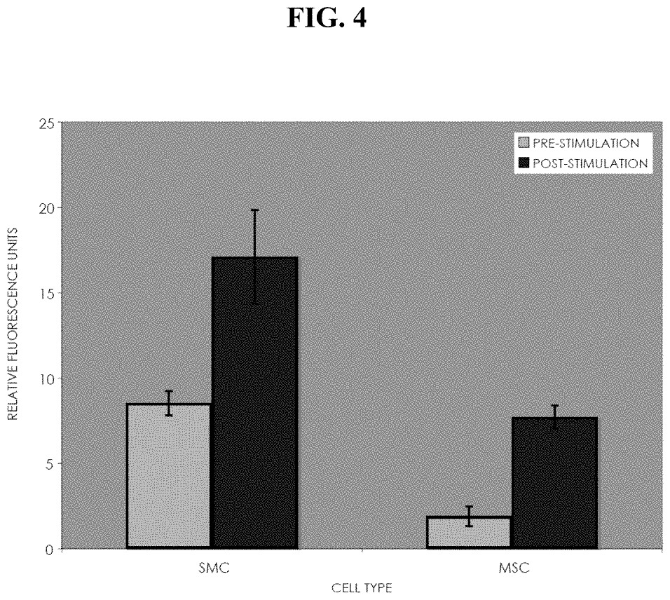

Carbachol stimulation resulted in a significant increase in relative fluorescence units (RFU) for both MSCs and SMCs with no significant difference between the two groups with regard to magnitude of increase (SMC: 8.6±2.5 RFU; MSC: 5.8±0.8 RFU)(SEE FIG. 4). Confocal microscopy demonstrated Ca²⁺ release accompanied by physical contraction of agonist stimulated MSCs (SEE FIG. 5). Subjective visualization indicates that MSCs can contract similarly to SMCs in vitro upon stimulation.

In vitro EPC Analyses

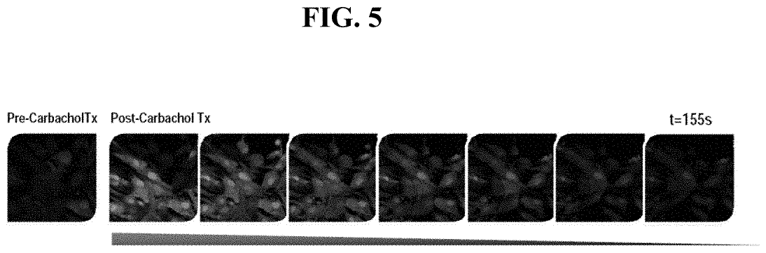

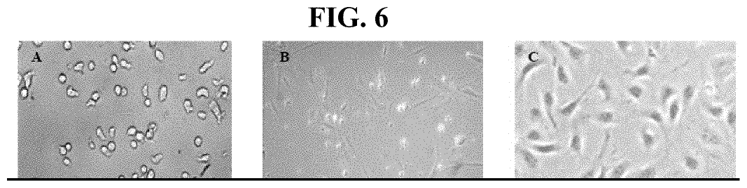

Day 14 flow cytometric analyses of endothelial markers on stimulated EPCs revealed approximately 55% of gated cells were von Willebrand Factor⁺ (vWF⁺) with 4% of cells CD31⁺. Furthermore, approximately 90% and 42% of the cells were integrinβ1⁺ and integrin αvβ3⁺, respectively (Table 1). Human umbilical vascular endothelial cells (HUVEC2s) grown in parallel displayed higher levels of vWF, CD31, and integrin αvβ3⁺ at day 14 of culture. The varied expression levels of these markers within the EPC population may be attributed to a lack of cellular maturation based upon inadequate time in culture. Morphological changes of EPCs demonstrated the transformation from spheroid suspension cells into spindle-like cells at day 14 in culture (SEE FIG. 6). These data indicate that EPCs have the potential of becoming endothelial cells in vitro.

### Chorioallantoic Membrane (CAM) Model

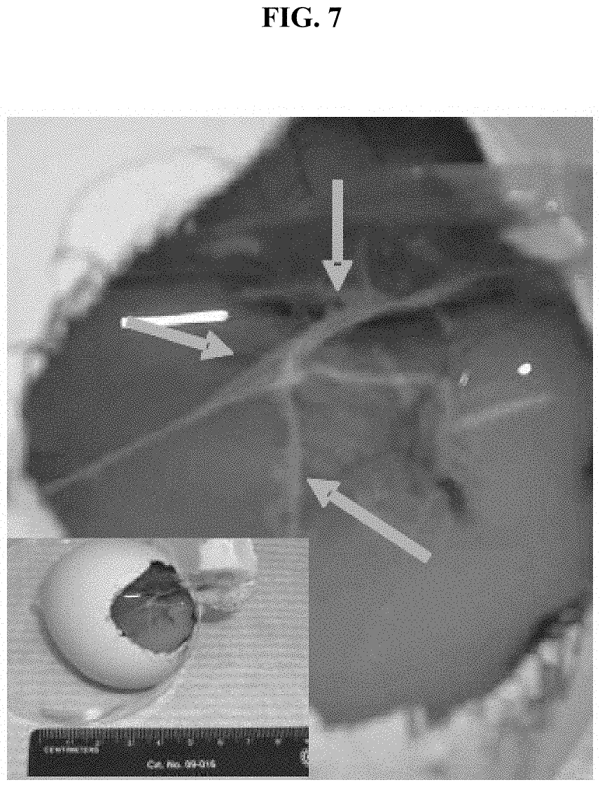

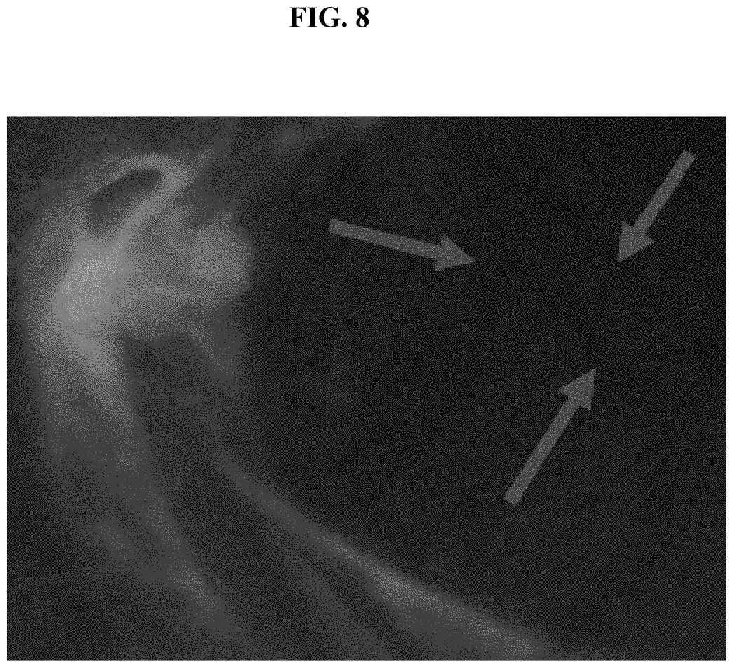

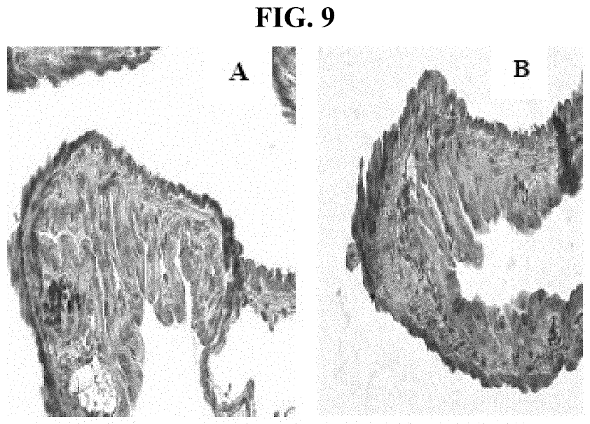

A GFP⁺ EPC seeded CAM model was assessed for EPC derived neo-angiogenesis. Egg seeded with GFP⁺ EPCs display a prominent green structure resembling vasculature at day 18 (SEE FIG. 7). This structure spanned the inner portion of the developing chick and branched off into several directions. The intense GFP signal displayed in white light emitted by the vessel-like structure can be attributed to the high constitutive, over-expression of GFP. A segment of this tissue was removed and placed under a fluorescence microscope, demonstrating GFP expression (SEE FIG. 8). Vessels in the background reveal that the fluorescence is not an artifact of autofluorescence. Further immunohistochemical analysis using the human specific MHC I antibody reveal positive staining demonstrating that the cells are of human origin (SEE FIGS. 9A and B) These data indicate that EPCs can form vasculature in vivo and may contribute to the overall vascularization of a newly reconstructed bladder.

### Example 3

### Lineage Commitment Potential of Spina Bifida (SB)-Derived Bone Marrow (BM) MSCs

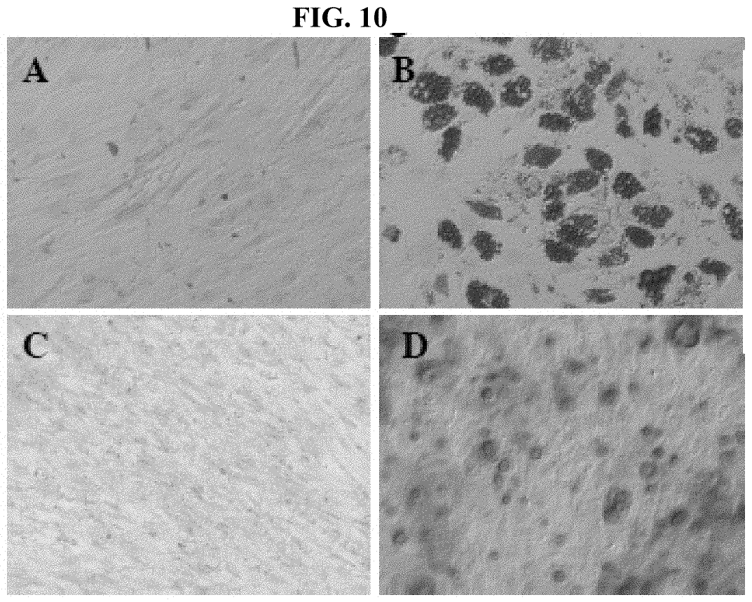

The developmental defects associated with SB adversely affect the physiological function of several organs. BM MSCs were isolated from SB patients. Epitope defined MSCs were then subjected to coerced terminal differentiation through standard assays. Isolated MSCs demonstrated the ability to differentiate into both adipocytes and osteocytes in vitro (SEE FIG. 10A-D). Both adipocytes and osteocytes are known lineages of coerced MSC differentiation. These experiments conducted during development of embodiments of the present invention demonstrate that the multipotential aspects of MSCs are not affected by SB and therefore can be utilized for bladder regenerative studies.

### Example 4

### Analyses of Cell Seeded Poly(1,8 octanediol-Co-Citrate) (POC) Films in a Bladder Augmentation Model

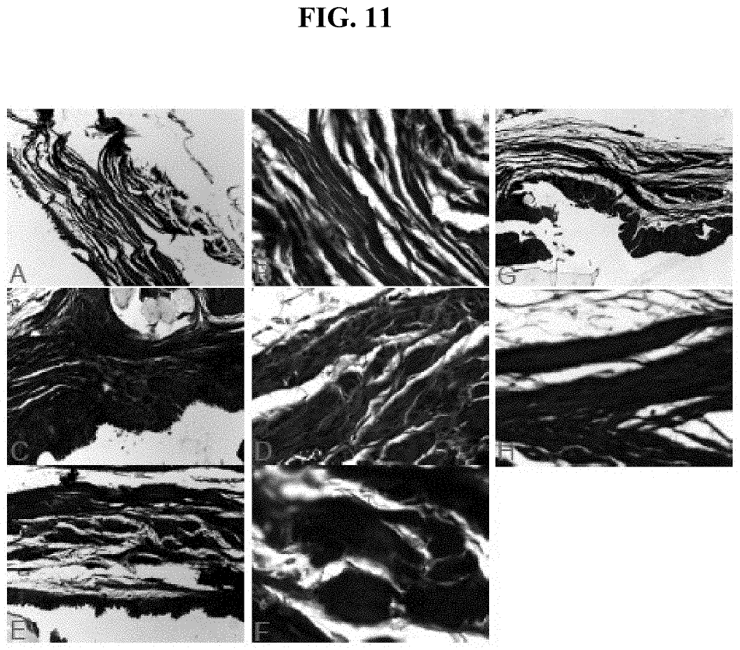

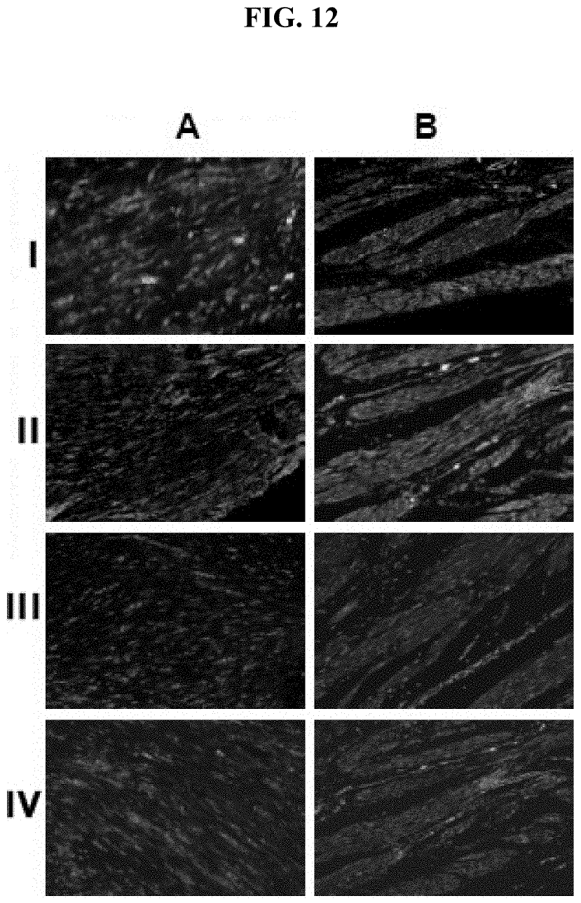

The capacity of BM derived MSCs from SB patients and their adult counterparts were evaluated in the nude rat bladder augmentation model. Studies indicated that animals augmented with MSC/Urotsa (an immortalized urothelial cell line) POC films from either adult or SB MSC sources grew equivalently or greater than normal bladder SMC cell control samples. Immunohistochemical (IHC) and histological quantification at 4 weeks post implantation was notable for a trilayered appearance consisting of urothelium, submucosa and muscle in both the adult and SB MSC and smooth muscle cell groups that developed along the surface of the POC film. Discrete muscle fasicles stained positive for human specific γ-tubulin and elastin, as well as calponin and caldesmon. The smooth muscle to collagen ratio was greatest for the SMC group (45/55%±11%) vs (34/66%±8%) and (12/88%±8%) in the MSC and POC alone groups, respectively (4 week IHC and histology data not shown). At 10 weeks, there was an increase in smooth muscle cell levels noted in the both the adult and SB MSC groups with a ratio of muscle to collagen 58/42%±11% and 64/36%±13% (for adult and SB MSC constructs, respectively) while there was a diminished ratio in the bladder SMC control groups (27/73%±11%) as demonstrated by trichrome staining (SEE FIG. 11). This corresponded to the appearance of comparably more robust trilayered architecture in both MSC groups at 10 weeks with muscle bundles staining positive for human specific γ-tubulin and elastin, as well as calponin and caldesmon indicating retention of human cells with their high regenerative capacity (SEE FIG. 12). The POC alone group demonstrated fibrosis and the absence of substantial muscle. Elastin expression in adult and SB MSC samples increased from the 4 to week time-points suggesting that bladder mechanics played role in the modulation of elastin, whose expression and function are a definitive requirement for bladder contraction and expansion as well as for further smooth muscle cell development.

### Example 5

### Characteristic Properties of POC and Cell Seeded POC

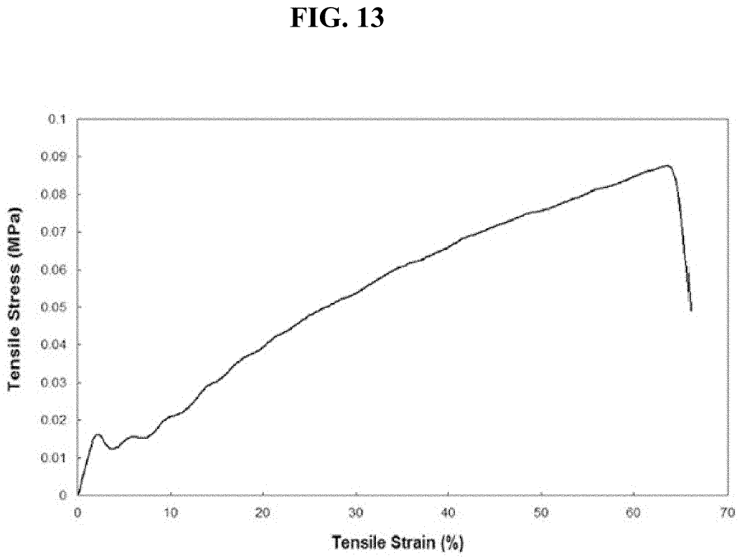

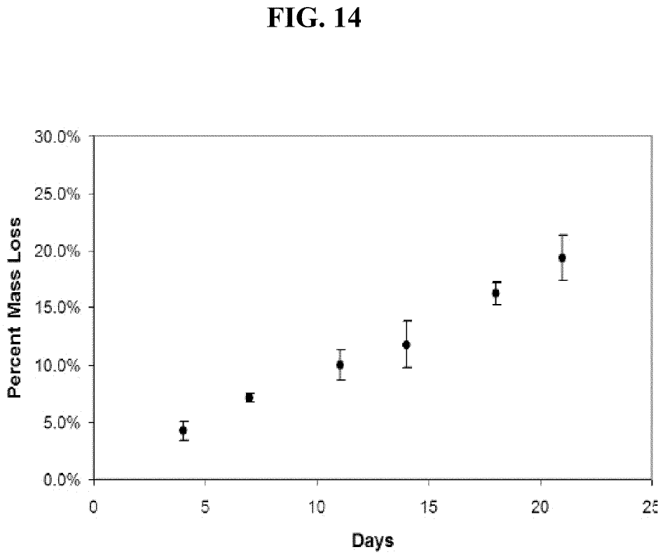

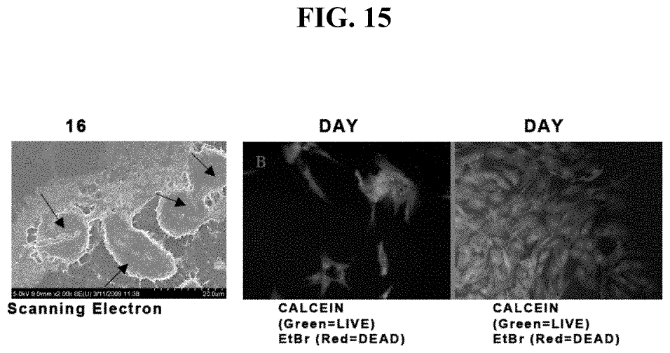

Synthesized POC films revealed a Young's modulus of 138.53 kPa±2.85 kPa as calculated by the slope of the tensile stress/strain data with the molecular weight between crosslinks equaling 72 kDa. (SEE FIG. 13). POC thin films were able to elongate to 137% of their initial length without permanent deformation demonstrating their high elastic potential. The potential of the POC thin film degraded with loss of mass over time (SEE FIG. 14).

### Example 6

### In vitro Growth Factor Release from POC Films

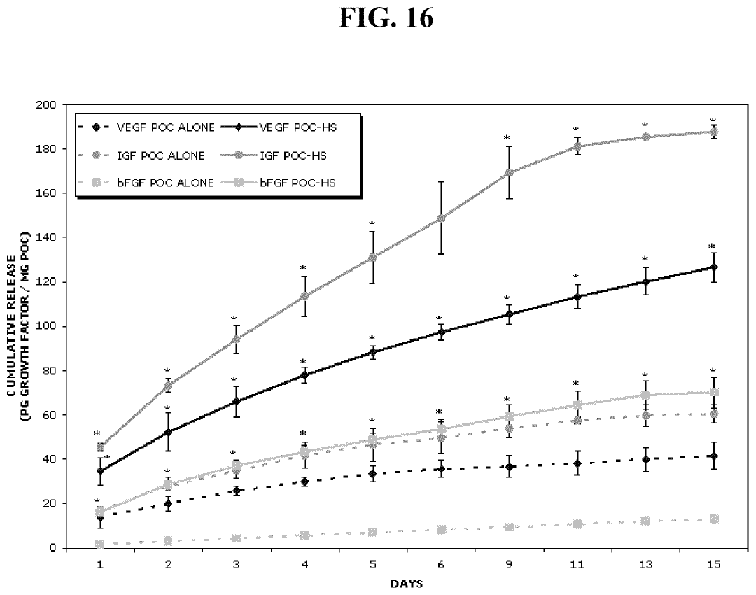

Several growth factors are vital to the growth and development of bladder tissue as a whole as well as the terminal differentiation of primitive endothelial progenitor cells (EPCs) into functional vasculature. The extended release of these growth factors in the immediate vicinity of implanted tissue constructs would provide an invaluable pillar for further cell maturation. POC films containing heparan can bind growth factors with complementary motifs found within pro-angiogenic molecules such as VEGF, IGF-1 and bFGF and release them over time. In vitro growth curves demonstrated that VEGF, IGF-1 and bFGF linked to heparan-bound POC (HBPOC) individually releases statistically significant levels of growth factor over a 15 day period (SEE FIG. 16) as compared to non-heparan bound POC control samples. These data demonstrate that growth factors can be released from HBPOC over extended periods of time and levels of growth factors can be subsequently be adjusted to custom fit levels of release.

### Example 7

### Urinary Bladder Regeneration using MSC-Seeded Elastomeric POC Thin Films

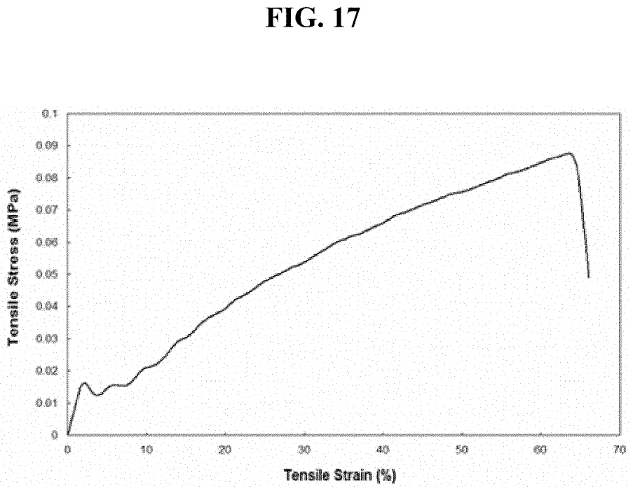

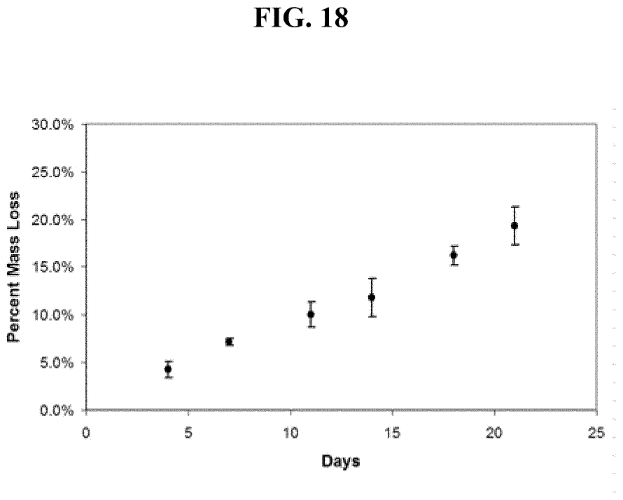

POC [poly(1,8-octanediol-co-citrate)] Thin Film Synthesis and Characterization Following the removal of unreacted monomers with several changes of protein free media, synthesized POCfs (POC films) were evaluated for their elastomeric potential by undergoing tensile mechanical stress evaluation. After being subjected to a mechanical tester, a Young's modulus of 138.53 kPa±2.85 kPa was obtained through the calculation of the slope of the tensile stress/strain data (SEE FIG. 17). Mechanical testing data demonstrated that POCfs possess ability to undergo elongation up to 137% of their initial length. The stresses endured by the POCf did not cause permanent deformation further demonstrating the high uniaxial elastic potential of the POCf, which is needed during rigorous contraction/expansion cycles of the bladder. A single batch of POC with an approximate modulus of 140 kPa was utilized throughout this study. In vitro degradation data of POCfs through a 22 day period displayed a linear mass loss trend. The best fit line was calculated using the sum of the least squares method from Excel 2007 (Microsoft Corporation, Redmond, Wash.). The best fit line equation utilized was: y=0.0088412244x+0.0052905939, x being the number of days, y being the percentage degradation (ranging from 0-1). Substituting in 1 for y (complete degradation) and back-calculating for x gave a total degradation time of approximately 113 days or an average weekly mass loss of 6.2±0.3%, which equates to approximately 16 weeks in vitro (SEE FIG. 18). The surface area of the disks used for degradation studies (0.47 cm²) was slightly greater the films used for augmentation (0.38 cm²). The films were grown in a larger volume to accommodate cellular growth.

### Characterization of POCfs and Cell Viability

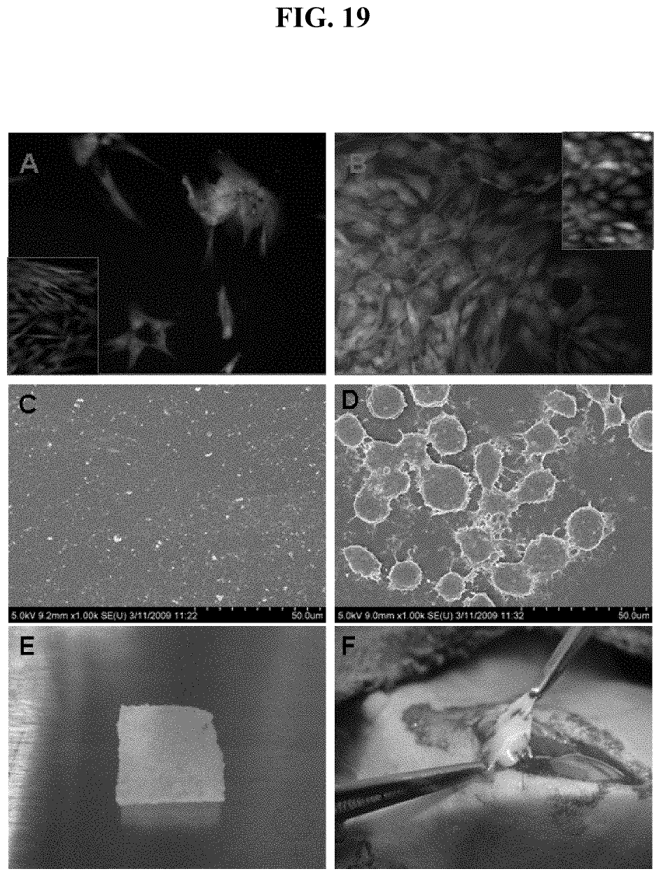

MSCs that were seeded upon POCfs exhibited typical fibroblast morphology after 1 day of seeding and showed no negative effect of being seeded onto the POCf as well as any indication of a cell death as determined by Calcein live/dead staining (SEE FIG. 19A). The procedural aspects of the Calcein live/dead staining allow for multiple staining of the cells without detriment to their viability. POCfs were deliberately seeded at a low density with MSCs so that the cells were allowed to grow into the POCf and proceed with rapid cellular expansion. This seeding regimen provided a robust means in which the extracellular matrix provided by the MSCs could be properly established onto the POCf. FIG. 19B depicts the same film at Day 21, and the viability at this time-point was calculated to be approximately 98%. These data demonstrate that POC is a non-toxic material suitable for the growth and proliferation of primitive bone marrow derived MSCs. Similarly, POCfs seeded with bladder SMCs and the Urotsa cell line grew normally with typical smooth muscle cell or urothelial cell morphology, respectively, and showed no deleterious side effects of being seeded and expanded on the POCfs (insets in SEE FIG. 19A and 19B, respectively). FIGS. 19C and 19D represent scanning electron micrographs (SEMs) of an unseeded POCf and an MSC seeded POCf 16 hours post seeding, respectively. The unseeded POCf is devoid of any remarkable characteristics while the seeded POCf displays MSCs in the early processes of cell attachment and extension which are in the initial phases of taking on fibroblast-like cell morphology. FIG. 19E depicts a typical POCf that was used for augmentation procedures. Although the POCf is an elastomer, it has the ability to hold its shape and can be molded to mimic a variety of shapes. FIG. 19F is a MSC/UC seeded POCf that has been augmented to the cystectomized rat bladder. FIG. 19F also demonstrates the ability of the POCf to stretch as it is being pulled upon by the forceps. The POCf is easily sutured without tearing or ripping, as seen by the suture entering into the bladder from the POCf.

### Histological Evaluation of Cell/POCf Composites and Urinary Tract Tissue

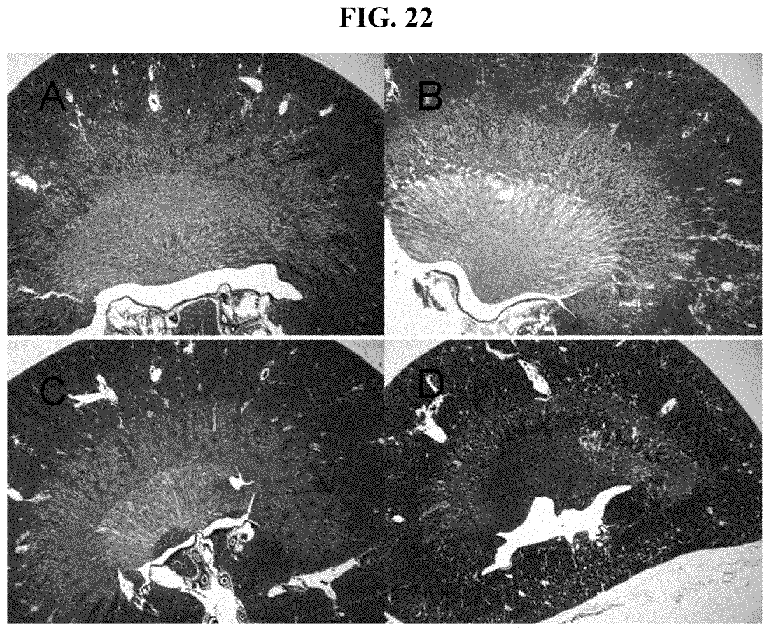

Explanted cell/POCf composites were subjected to histological characterization at 4 and 10 weeks post implantation. At the 4 week time-point, Trichrome staining revealed a trilayered appearance consisting of urothelium, submucosa and muscle in both the SMC/UC and MSC/UC POCf groups (SEE FIG. 20A and 20B respectively, 10× mag). Unseeded POC samples exhibited a thin layer of UCs adjacent to a fibrotic submucosa that was devoid of muscle (SEE FIG. 20C, 10× mag). The urothelium layer that is depicted in FIGS. 20A-C did not stain positive with the human specific γ-tubulin antibody. Control slides containing human bladder tissue stained with γ-tubulin antibody reveal positive staining in the smooth muscle compartment as well as the urothelium layer of the bladder, indicating that the endogenous rat urothelium grew between the POCf and the MSC or SMC seeded side of the POCf. FIGS. 20D and E (40× mag of images 20A and B, respectively) depict the organized distribution of muscle and collagen amongst SMC and MSC seeded samples while augmented unseeded POCfs consisted mainly of collagen based tissue indicating the possibility of heavy scar tissue formation (SEE FIGS. 4C,F). Upon resection of augmented bladders from the animals, unseeded POCf samples were consistently rigid and exhibited fibrosis on both the luminal and extra-luminal sides of the bladder. SMC and MSC seeded composites remained robust, pliable and retained their elastic characteristics. At the 10 week time-point, the SMC/UC POCf group displayed less organized SMC formation compared to the MSC/UC POCf group (SEE FIGS. 21A and B). There was a marked increase in smooth muscle formation noted in the MSC/UC POCf augmented group that was accompanied by very robust trilayered architecture. Unseeded POCfs consistently remained devoid of smooth muscle tissue (SEE FIGS. 21C and F). Higher magnification of FIGS. 21A and B demonstrate the organization of the muscle component of the SMC/UC POCf and MSC/UC POCf composites into discrete muscle fascicles (SEE FIGS. 21D and E). This smooth muscle bundle formation was not evident at the 4 week time-point and this data suggests that the maturation period from the 4 to 10 week time-point allowed for the specific organization of the implanted cells into smooth muscle bundles that are reminiscent of native bladder smooth muscle tissue. The upper urinary tracts (ureters and kidneys) of animals that underwent augmentation cystoplasty were examined and routinely demonstrated no evidence of upper tract dilatation at 4 and 10 weeks post implantation in approximately 98% of the animals. This is consistent with a non-obstructive lower urinary tract and maintenance of low pressure physiologic bladder filling and unobstructed voiding. There was a small subset with significant stone formation. The ureters in these animals were noted to be dilated to the level of ureteropelvic junction without evidence of gross renal pelvis dilatation. FIGS. 22A-D depict the right kidney that was removed from either POCf alone, SMC/UC POCf, and MSC/UC POCf augmented animals as well as a normal rat kidney specimen, respectively. Data depicted in this image shows no gross morphological differences between calculi free augmented animals and calculi free normal animal kidneys suggesting that POC had no detrimental effects on the lower and upper urinary tract.

### Quantitative Morphometry of Stained Cell/POCf Composites

The smooth muscle to collagen ratio determined by Trichrome staining was evaluated at both 4 and 10 week time-points. 4 week data reveals that the SMC/UC POCf groups had a 49.43%±9.48% muscle:collagen ratio versus 38.88%±9.51% for the MSC/UC POCf groups and a 21.97%±9.60% for the unseeded POCf groups. As the duration of tissue maturation in vivo increased to the 10 week time-point, there was shift in expression of muscle and collagen in both of the cell seeded groups. MSC/UC POCf composites demonstrated an increase in expression of muscle to 55.96%±9.81%, an increase of approximately 1.4 fold. SMC/UC POCf composites decreased muscle content approximately 1.5 fold falling to 31.73%±7.98%. Unseeded POCf groups remained mostly collagen based with a ratio of 16.77%±4.72%. This data is further suggestive of utilizing MSCs as an alternative cell source for smooth cell replacement in the bladder. FIG. 23 depicts cell/film composites and unseeded POCfs at 4 and 10 week time-points where data is represented as percent muscle.

### Immunofluorescent Imaging of Augmented Cell/POCf Composite Tissues

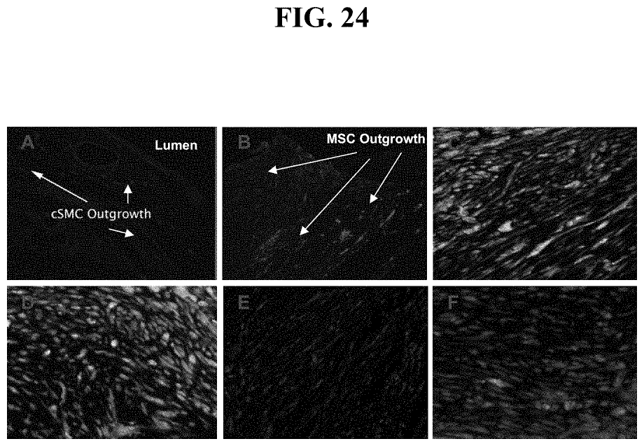

The capacity of bone marrow derived MSCs to regenerate the smooth muscle component of the urinary bladder wall was evaluated in the nude rat bladder augmentation model at 4 and 10 weeks post augmentation cystoplasty. 4 week tissue samples were stained with antibodies against smooth muscle epitopes that are essential for smooth muscle contraction and expansion. FIG. 24A shows the presence of cSMC out-growth on the extra-luminal side of the augmented bladder, while FIG. 24B depicts that presence of MSCs that have been stained with human specific γ-tubulin conjugated to Alexa 555 (γ-tubulin does not cross react with rat bladder tissue). The MSCs appear to be concentrated adjacent to the luminal side of the POCf but also extend throughout the graft within the anastomosed region of the bladder and appear to be fairly abundant. MSCs were not found outside the region of augmentation. Similarly, FIGS. 24C and D were dual stained with either γ-tubulin/calponin and γ-tubulin/caldesmon, respectively. The resulting orange colored overlay suggests the presence of human specific cells that express smooth muscle contractile proteins. FIG. 24E demonstrates the abundant staining of elastin, again utilizing an antibody that is human specific. FIG. 24F shows positive staining of smooth muscle γ-actin combined with γ-tubulin that again resulted in an orange overlay. A second group of animals was sacrificed at the 10 week time-point and tissue samples were subjected to the same battery of antibodies and displayed similar staining patterns. FIG. 25A demonstrates a section of a MSC/UC POCf exhibiting robust smooth muscle bundle formation which is positive for γ-tubulin suggesting the bundles are of human origin. Data also demonstrates that MSCs have the capacity to form the typical architecture found in native bladder tissue as compared to bladder SMCs. This is further illustrated in FIG. 25B in which discrete muscle fascicles stained positive for human specific γ-tubulin as well as γ-actin. FIGS. 25C-E demonstrate the retention of SMC markers caldesmon, elastin and calponin, respectively, in which the caldesmon and calponin samples have also been stained with γ-tubulin at the 10 week juncture. Observation of the tissue sections stained with antibodies demonstrates that the expression level of each epitope appears to increase over time when comparing 4 and 10 week time-points suggesting that the MSCs are continually growing and adding to the overall architecture of the bladder. Due to the high constitutive over-expression of the mCherry gene product in transduced bladder SMCs, immunofluorescent antibody staining with the aforementioned antibodies was not possible due to the high levels of fluorescent bleed through in all visualizing microscopic channels. However, it has been demonstrated that identical bladder SMCs do express the aforementioned smooth muscle epitopes at the protein level (Sharma et al. J Urol 2009;182(4 Suppl):1898-1905.; herein incorporated by reference in its entirety). This data demonstrate that MSCs may be used as an alternative cell source for bladder smooth muscle cell regeneration.

All publications and patents mentioned in the present application and/or listed below are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A composition comprising a scaffold and a cell mixture, said mixture comprising isolated bone marrow stem and progenitor cells.

2-10. (canceled)

11. A method for transplanting stem cells in vivo, comprising:
a. providing:
i. a host organism;
ii. isolated bone marrow stem and progenitor cells; and
b. transplanting said bone marrow stem and progenitor cells into a tissue of said host.

12-19. (canceled)

20. A method for urinary bladder regeneration in a subject comprising transplanting mesenchymal stem cells and endothelial progenitor cells onto existing bladder tissue in the subject, wherein the mesenchymal stem cells are CD29+, CD44+, CD105+, and CD169+ and are at least 98% pure, and wherein the endothelial progenitor cells are CD34+ and CD133+ and are at least 98% pure.

21. The method of claim 20, wherein the existing bladder tissue is injured or diseased tissue.

22. The method of claim 21, wherein the existing bladder tissue is injured as the result of trauma.

23. The method of claim 21, wherein the subject has bladder cancer.

24. The method of claim 21, wherein the subject has spina bifida.

25. The method of claim 20, wherein the mesenchymal stem cells and endothelial progenitor cells are autologous to the subject.

26. The method of claim 20, wherein the mesenchymal stem cells and endothelial progenitor cells can be differentiated.

27. The method of claim 26, wherein the mesenchymal stem cells and endothelial progenitor cells differentiate into bladder tissue in the subject.

28. The method of claim 20, wherein the mesenchymal stem cells and endothelial progenitor cells scaffold, and the scaffold is transplanted onto the existing bladder tissue in the subject.

29. The method of claim 28, wherein the scaffold is a thin-film scaffold.

30. The method of claim 29, wherein the thin-film scaffold comprises poly(1,8 octanediol-co-citrate) (POC).

31. The method of claim 20, wherein the mesenchymal stem cells and endothelial progenitor cells comprise adult mesenchymal stem cells and endothelial progenitor cells.

32. The method of claim 20, wherein the mesenchymal stem cells and endothelial progenitor cells comprise pediatric mesenchymal stem cells and endothelial progenitor cells.
